(19) United States
(12) Patent Application Publication
(10) Pub. No.: US 2005/0081256 A1
(43) Pub. Date: Apr. 14, 2005

(54) Sex- specific selection of sperm from transgenic animals

(76) Inventors: Erik J. Forsberg, Oregon, WI (US); Kenneth J. Eilertsen, Baton Rouge, LA (US); Ying Zheng, Wayne, PA (US); Gregory H. Leno, Madison, MI (US)

Correspondence Address: FOLEY & LARDNER, P.O. BOX 80278, SAN DIEGO, CA, 92138-0278, US

(21) Appl. No.: 10/472,542

(22) PCT Filed: Mar. 22, 2002
(86) PCT No.: PCT/US02/08933

Related U.S. Application Data
(60) Provisional application No. 60/278,155, filed on Mar. 22, 2001.

(51) Int. Cl. A01K 67/027

(52) U.S. Cl. 800/14; 800/15; 800/16; 800/17; 435/325

(57) ABSTRACT

The present invention relates to methods and materials for pre-selecting the sex of mammalian offspring. In particular, the materials and methods described herein permit the enrichment of X- or Y-chromosome-bearing sperm in semen by introducing a transgene into a sex chromosome under control of regulatory sequences that provide for expression of the transgene in a haploid-specific manner.

The present application claims priority to U.S. Provisional Patent Application No. 60/278,155, filed on Mar. 22, 2001, which is hereby incorporated by reference in its entirety, including all tables, figures, and claims.

### FIELD OF THE INVENTION

The present invention relates to methods for pre-selecting the sex of mammalian offspring. In particular, the materials and methods described herein permit the enrichment of X or Y chromosome-bearing sperm in semen by expressing a transgene present on a sex chromosome in a haploid-specific manner.

### BACKGROUND OF THE INVENTION

Throughout history, humans have sought the ability to assert control over the sex of offspring; both human and livestock. Homo sapiens' attempts to select sex of offspring prior to conception has been well-documented, as evidenced by historical descriptions of methods. Early techniques, circa 500 B.C., began with monoorchydectomy and progressed through a variety of techniques which have come down to us via folklore (such as placing an egg or scissors under the bed for conception of a girl, and placing a hammer under the bed and tying off the left testicle to conceive a boy) (Fugger, 1999, Theriogenology 52:1435-1440). A more scientific approach began in the last century and included utilizing a reported differential survival between X and Y spermatozoa dependent on the pH of the medium. (Shettles, 1970). Further techniques progressed to exploit differences in motility (Ericsson et al., 1973, Nature 246:241-24, Steeno et al., 75, Botchan et al., 1997) or cell density (e.g., centrifugation in a Percoll gradient, Lin et al., 1998, J. Assist. Reprod. and Genetics 15:565-569) to use in distinguishing X from Y sperm. Other techniques tried include size, head shape, surface properties, surface macromolecules, mass, and swimming velocity (see review by Windsor et al., 1993, Reprod. Fert. Dev. 5:155-71). One group, Fabricant et al., (U.S. Pat. No. 4,722,887), utilized the differential expression of a sperm cell-surface sulfoglycolipid to develop a method for separating X-chromosome-bearing and Y-chromosome-bearing sperm by polymeric phase separation.

A recent approach to the problem of sex pre-selection relates to methods that rely on the use of antibodies directed to sex-specific epitopes on sperm, or, alternatively, on fertilized embryos. For example, evidence for a male-specific cell surface antigen was first obtained by Eichwald and Silmser (1955, Transplant Bull 2:148) using the inbred mouse strain C57BL/6, but it remained for Hauscha (Transplant Bull, 1955, 2:154) to later hypothesize the existence an antigen coded for by a Y-linked gene. This surface marker became known as H-Y (bistocompatibility locus on the Y chromosome). Y-sperm-specific surface expression of the H-Y antigen has been suggested to be a target epitope for sex pre-selection, and antibodies raised to the H-Y antigen were expected to allow the routine sorting of sperm using cell sorting or immunological adsorption of H-Y expressing sperm (Peter et al., 1993, Theriogenology 40:1177-1185). Similarly, sex-specific antibodies were disclosed as allowing the selective ablation of sperm or embryos utilizing complement (U.S. Pat. No. 5,840,504). See also, U.S. Pat. No. 4,999,283; U.S. Pat. No. 4,511,661; U.S. Pat. No. 4,191,749; U.S. Pat. No. 4,448,767; U.S. Pat. No. 4,680,258; and U.S. Pat. No. 5,840,504.

The locus of at least one of the genes responsible for H-Y expression is on the Y chromosome, and this antigen has been shown to be cross-reactive among numerous speciess ranging from fish to man. It is possible that the H-Y antigen may be the primary sex determinant and may control testicular development in mammals. (Wattle, et al., 1975; Wattle and Ok, 1980); Ok, et al., “Application of Monoclonal Anti-H-Y Antibody for Human H-Y Typing,” Human Genetics, 57: 64-67 (1981). H-Y is a “minor” histocompatibility antigen, which is a separate genetic locus from the major histocompatibility complex (MHC). Minor histocompatibility loci are mainly concerned with cellular immunity; few if any products of these loci are efficient in raising antibodies. Nevertheless, a search for a serological counterpart to the transplantation H-Y antigen appeared to have been successful when a serological “E-Y” method was reported by Goldberg and coworkers (1971, Nature 232: 478). Recent data indicates, however, that the serological detectable “H-Y” antigen may not be the same as the histocompatibility antigen. (Simpson et al., 1990, Arch. Androl. 24:235). The molecule identified by serological methods is now widely referred to as serologically detectable male antigen (SMA).

These immunological methods have not always lived up to expectations however (Bradley, 1989). For example, some authors found no evidence that H-Y is preferentially expressed on Y-bearing sperm (e.g. Hendricksen et al. 1993, Mol. Reprod. Devel. 35:189) and, in a review, Windsor et al. (1993, Reprod. Fert. Dev. 5:155) have concluded that no differences between the two classes of sperm can be detected immunologically.

Another method recently described as showing utility for sex pre-selection involves the use of Fluorescence Activated Cell Sorting (FACS) for sorting sperm based on the reduced amount of DNA in Y sperm as opposed to X sperm due to the small mass of the Y chromosome. The difference in DNA content between X and Y sperm, ranges from 2.8% in humans and 4.0% in most livestock, to 12.5% in voles (Gillis, 1995). See, e.g. Rath et al., 1999, J. Anim. Sci. 77:3346-3352; Welch and Johnson, 1999, Theriogenology 52:1343-1352; Fugger et al., 1998, Human Reprod. 13: 2367-2370; Cran et al., 1995, Vet. Rec. 135: 495-496; Seidel et al., 1997, Theriogenology 48: 1255-1265.

FACS sorting, following by insemination, has been shown to work in bulls, rams (Johnson and Clark, 1988) and humans (Johnson et al., 1993). In spite of these successes, this technique is limited by three factors. First, it requires the sophisticated operation of expensive machines. Second, the reagents used to fluorescently label the DNA and the near UV light used to detect the dyes may lead to chromosomal damage and/or mutations. Third, this technique has a poor yield. Progress in these techniques has recently been summarized in review articles by Reubinoff and Schenker (1996) and Botcham et al (1997).

In another example, which combines sorting based on DNA content, followed by immunological selection, Spaulding, (U.S. Pat. Nos. 5,021,244 and 5,346,990, and 5,660,997) first sorted sperm into enriched X- and Y-chromosome bearing preparations via DNA content and cell sorting techniques. Spaulding then used the sorted sperm to screen for sex-specific sperm proteins and then proceeded to predict the use of the sex-specific protein for raising antibodies to allow purification of the sperm population to either X-chromosome bearing or Y-chromosome bearing populations.

WO 01/47353 proposes methods by which expression of a transgene inserted into a sex chromosome might alter the sex ratio of offspring.

The dairy industry demands a large number of females cows for the production of milk, and currently male calves, except those necessary for breeding, are culled. Similarly, for the production of beef, male cattle are preferred. In spite of recent progress in techniques for sorting male sperm (Y) from female sperm (X), the techniques still lack the robustness needed for routine use for the commercial production of livestock. One reason is that the techniques available are difficult to use to produce the large numbers of viable spermatozoa required for use in the production of livestock. Also, some of the techniques carry with them the threat of creating mutations while sorting sperm. Thus, there remains a need in the art for methods and materials permitting the sex pre-selection of offspring.

### SUMMARY OF THE INVENTION

The present invention discloses a robust technique for producing semen that is enriched for active sperm containing either the X chromosome or the Y chromosome. Because cows of reproductive age normally will give birth to only a single calf per year, which will randomly either be male or female, the ability to pre-select the sex of an offspring is particularly advantageous for the dairy and meat industries. However, in the agricultural industry generally, methods for sex selection could be used to upgrade the nutritional characteristics and quantities of animals produced. Accurate selection of the sex of the offspring could allow the birth of many genetically superior animals of a single sex as offspring of one genetically desirable parent. Thereby, the desirable genetic characteristics of the parent animals can be propagated with much greater velocity than is possible in nature. The ability to increase the reproductive capacity of genetically prized animals, especially dairy cattle, may be a key to solving the hunger problem which exists in many countries today by allowing a more efficient use of available resources.

In a first aspect, this invention relates to animals in which one or more transgenes are incorporated into either the X or Y chromosome, and hence into those sperm cells containing a specific sex chromorome, of the transgenic animal. Preferably, the transgene(s) is (are) under the control of a promoter region and/or an enhancer region which is capable of conferring haploid-specific expression to the coupled trausgene. In these embodiments, the semen produced by the transgenic animal can be enriched for sperm of a given-sex by expression of the transgene.

Transgenes useful for this invention include genes that encode a gene product which is toxic for a haploid cell when expressed in cis, e.g., suicide genes such as pertussis toxin or the immunoglobulin heavy chain binding protein (BiP); alternatively, gene products that allow for survival in cis when the sperm cell is exposed to a selective agent may be employed. The term “in cis” is defined hereinafter. In other embodiments, the gene may encode an antisense construct capable of blocking the expression of a gene essential for the continued viability or function of the sperm.

The only requirement of the transgene(s) used in the instant invention is that they may be expressed in a haploid-specific manner, and that transgene expression results in enhanced production of offspring having the selected sex. The transgenes of the instant invention need not result in the death of the haploid cells in which it is expressed, however, in order to enrich for sperm of a selected sex. For example, a gene may prevent induction of pregnancy by a haploid cell, for example by preventing fusion of a sperm with an oocyte, or by reducing or preventing motility. Even a minor change in fitness, resulting from the presence of one or more transgenes, may result in enhanced production of offspring having the selected sex. See, e.g., Ellison et al., Mol. Reprod. Dev. 55: 249-55 (2000).

The transgenes of the instant invention may also encode gene products that allow the haploid cells expressing the gene to be detected by a detection method, e.g., optically. Genes which can be detected optically include the Green Fluorescent Protein (GFP) (Tsien, 1998, Annu. Rev. Biochem. 67:509-44), drFP83 and the ES mutant (Terskikh, et al., 2000, Science 290:1585-1588).

Finally, the transgenes of the instant invention may encode gene products that make a haploid cell apparent to an in vivo immune response. For example, sex chromosome-specific immune infertility may be produced by immunizing an animal against a transgene product expressed in a sex chromosome-specific and haploid-specific manner. Such immunity may be created in either a male or a female, resulting in enhanced production of offspring of the selected sex. See, e.g. Tsuji et al., J. Reprod. Immunol. 46: 31-8 (2000); Mahmoud et al., Andrologica 28: 191-6 (1996).

The term “haploid cell” as used herein refers to cells that contain a single set of unpaired chromosomes. In animals, cells that give rise to gametes (i.e., sperm and eggs) undergo meiotic division, whereby a diploid cell divides into four haploid cells. In males, a diploid cell contains both an X and a Y chromosome, referred to herein as “sex chromosomes.” Each haploid cell contains only one sex chromosome. The term “haploid cell” can preferably refer to the following cells produced by a male animal: primary spermatocytes (produced in the first meiotic division); secondary spermatocytes (produced in the second meiotic division); spermatids; differentiating spermatids; and spermatozoa. The term “haploid cell” can also refer to cells produced by a female animal, e.g., oocytes and eggs.

The term “transgenic” as used herein refers to a cell or an animal that comprises heterologous deoxyribonucleic acid (DNA). Methods for producing transgenic cells and animals are well known to the ordinarily skilled artisan. See, e.g., Mitani et al., 1993, Trends Biotech, 11: 162-166; U.S. Pat. No. 5,633,067, “Method of Producing a Transgenic Bovine or Transgenic Bovine Embryo,” DeBoer et al., issued May 27, 1997; U.S. Pat. No. 5,612,205, “Homologous Recombination in Mammalian Cells,” Kay et al, issued Mar. 18, 1997; and PCT publication WO 93/22432, “Method for Identifying Transgenic Pre-Implantation Embryos;” Kereso et al., 1996, Chromosome Research 4: 226-239; Holló et al., 1996, Chromosome Research 4: 240-247; U.S. Pat. No. 6,025,155, and U.S. Pat. No. 6,077,697; all of which are incorporated by reference herein in their entirety, including all figures, drawings, and tables.

The term “heterologous DNA” refers to DNA having (1) a different nucleic acid sequence than DNA sequences present in cell nuclear DNA; (2) a subset of DNA having a nucleotide sequence present in cell nuclear DNA, where the subset exists in different proportions in the heterologous DNA than in the cell nuclear DNA; (3) a DNA sequence originating from another organism species than the species from which cell nuclear DNA originates; and/or (4) a different nucleic acid sequence than DNA sequences present in cell mitochondrial DNA. An artificial chromosome present in a transgenic cell can comprise heterologous DNA. Heterologous DNA can encode multiple types of recombinant products, as defined hereafter.

The term “different nucleic acid sequence” as used herein refers to nucleic acid sequences that are not substantially similar. The term “substantially similar” as used herein in reference to nucleic acid sequences refers to two nucleic acid sequences having preferably 80% or more nucleic acid identity, more preferably 90% or more nucleic acid identity or most preferably 95% or more nucleic acid identity. Nucleic acid identity is a property of nucleic acid sequences that measures their similarity or relationship. Identity is measured by dividing the number of identical bases in the two sequences by the total number of bases and multiplying the product by 100. Thus, two copies of exactly the same sequence have 100% identity, while sequences that are less highly conserved and have deletions, additions, or replacements have a lower degree of identity. Those of ordinary skill in the art will recognize that several computer programs are available for performing sequence comparisons and determining sequence identity.

A “transgenic animal” is an animal having cells that contain DNA which has been artificially inserted into a cell, which DNA becomes part of the genome of the animal which develops from that cell. Preferred transgenic animals are mammals, most preferably non-human primates, mice, rats, ungulates (including cows, pigs, horses, goats, and sheep), dogs and cats. Preferably, a transgenic animal expresses one or more gene products in a haploid-specific manner. Additionally, preferred sites of integration of a heterologous DNA in a transgenic animal of the instant invention include the Y chromosome and the X chromosome.

Numerous methods are well known in the art for producing transgenic animals. For example, a nucleic acid construct according to the invention can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82:4438-4442, 1985). Alternatively, embryos can be infected with viruses, especially retroviruses, modified to carry nucleic acid constructs according to the invention, or other gene delivery vehicles. In particularly preferred embodiments, transgenic animals can be produced by nuclear transfer using a transgenic nuclear donor cell. Nuclear transfer methods are well known to the ordinarily skilled artisan, and are described in detail hereinafter. See, e.g., U.S. Pat. No. 6,107,543; U.S. Pat. No. 6,011,197; Proc. Nat'l. Acad. Sci. USA 96: 14984-14989 (1999); Nature Genetics 22: 127-128 (1999); Cell & Dev. Diol 10: 253-258 (1999); Nature Biotechnology 17: 456-461 (1999); Science 289: 1188-1190 (2000); Nature Biotechnol. 18: 1055-1059 (2000); Nature 407: 86-90 (2000).

The term “transgene” refers to the heterologous DNA included in a transgenic cell or animal. The transgene may refer to the coding sequence or it may also refer to the coding sequence plus additional 5′ and 3′ DNA sequences necessary for the proper expression of the transgene. A cell may contain multiple transgenes, which may or may not be identical to one another.

The term “expression” as used herein refers to the production of the protein encoded by a transgene useful in the invention from a nucleic acid vector containing protease genes within a cell. The nucleic acid vector is transfected into cells using well known techniques in the art as described herein. The nucleic acid vector is preferably integrated into the genome of the host.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which directs the initiation of RNA transcription. Such regions will also normally include those 5′-non-coding sequences involved with initiation of transcription and translation, such as the TATA box, capping sequence, CAAT sequence, and the like.

The term “promoter” as used herein, refers to nucleic acid sequence needed for gene sequence expression. Promoter regions vary from organism to organism, but are well known to persons skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like. In preferred embodiments, a promoter is sex-specific, and/or sperm-specific, -and/or inducible. A particularly preferred promoter is the protamine promoter.

The term “sex chromosome-specific expression” refers to expression of a gene product in cells with a specific sex chromosome. Particularly preferred is sex chromosome-specific expression in haploid cells, which, by definition, contain only a single sex chromosome. Sex chromosome-specific expression of a gene can be achieved by inserting the gene to be expressed into the specific sex chromosome. In preferred embodiments, a gene is rendered X chromosome-specific by its operable incorporation into the X chromosome. In these embodiments, only haploid cells that contain an X chromosome will exhibit expression of the gene product. In a similar fashion, a gene may be rendered Y chromosome-specific by its operable incorporation into the Y chromosome.

The term “haploid-specific expression” refers to expression of a gene product only by haploid cells, such as spermatozoa, spermatids, etc. The gene product may be expressed during assembly, during spermatogenesis, or after at any time prior to fertilization. In particularly preferred embodiments, a gene that is expressed in a haploid-specific fashion is also expressed in a sex chromosome-specific fashion.

The transgenes of the instant invention may also be configured and arranged to confer “tissue-specific” expression on the transgene. That is, the expression of the transgene may take place only in specific body tissue(s) of the transgenic animal. Particularly preferred are transgenes that are expressed only in the testis or only in the ovary of the transgenic animal.

The term “specific expression” refers to gene expression that is predominantly localized to a desired cell type. Such expression may be “leaky,” i.e., there may be some ectopic expression of the gene in undesired cell types, but the predominant expression may still be in the specific cell type. In preferred embodiments, “specific expression” refers to a gene that is expressed 5-fold higher, 10-fold higher, 20-fold higher, 50-fold higher, and 100-fold higher or more in the desired cell type when compared to expression in undesired cells.

Regulatory sequences that may provide for haploid-specific expression and/or tissue-specific expression are well known to the skilled artisan. See, e.g., Yamanaka et al., Biol. Reprod. 62: 1694-1701 (2000); Westbrook et al., Biol. Reprod. 63: 469-81 (2000); Tosaka et al., Genes Cells 5: 265-76 (2000); Reddi et al., Biol. Reprod. 61: 1256-66 (1999); Nayernia et al., Biol. Reprod. 61: 1488-95 (1999); Mohapatra et al., Biochem. Biophys. Res. Comm. 244: 540-5 (1998); Herrada et al., J. Cell Sci. 110: 1543-53 (1997); Rodriguez et al., J. Androl. 21: 414-20 (2000); and Lee et al., Biol. Chem. Hoppe Seyler 368: 807-11 (1987). In preferred embodiments, the gene that is expressed in a haploid-specific manner is under the control of the promoter of the protamine gene. See, e.g., Queralt and Olivia, Gene 133: 197-204 (1993).

In certain preferred embodiments, the transgene is capable of killing haploid cells in which it is expressed (“in cis”) and not in cells not expressing the transgene; while in other preferred embodiments, the transgene is capable of functionally disabling haploid cells in cis when expressed.

The term “killing haploid cells” refers to the ability of one or more expressed gene products to kill a haploid when expressed. The gene(s) may kill the haploid either directly though the activity of one or more expressed proteins, or indirectly, via metabolizing an exogenously supplied compound to produce a toxic product or by failing to metabolize a toxic chemical supplied exogenously. In preferred embodiments, the gene product(s) are expressed in a haploid-specific manner; in other embodiments, the gene product(s) are expressed in an inducible fashion. Particularly preferred as a gene to kill haploid cells is the immunoglobulin heavy chain binding protein (BiP) gene, mutations of which have been shown to exhibit dominant negative effects in cells. See, e.g., Hendershot et al., Proc. Natl. Acad. Sci. USA 93: 5269-74 (1996).

The skilled artisan will recognize that expression of a gene may also render haploid cells in which it is expressed viable in the presence of a molecule that would ordinarily kill or disable the cells. Such a strategy is often used, e.g., by inserting antibiotic resistance genes into cells, then killing those cells that do not express the resistance gene by contacting the cells with an antibiotic.

The term “disabling haploid cells” refers to the ability of one or more expressed gene products to prevent the proper functioning of a haploid cell when expressed, without killing the cell. Genes which may disable haploid cells include, but are not limited to, (1) proteins that disturb ionic gradients by forming pores in the membranes of a cell, both extracellular and intracellular, (2) proteins that interfere with the motility of sperm, e.g., by binding to microtubules, by affecting protein tyrosine kinases, etc., (3) enzymes capable of degrading DNA such as those involved in apoptosis, (4) proteins that are directly toxic to the cell, (5) enzymes that produce a compound which is toxic to the cell when supplied with an exogenous metabolite, and (6) proteins that affect energy metabolism. The term “disabling” can also refer to acting upon a haploid cell so as to reduce or destroy its mobility, to disrupt or degrade its DNA so as to block the ability of the DNA to be used in creating a viable offspring, or to prevent it from binding to and combining with another haploid cell (i.e., participating in fertilization). See, e.g., Uma Devi et al., Andrologia 32: 95-106 (2000); Jelks et al., Reprod. Toxicol. 15: 11-20 (2001); Jones & Bavister, J. Androl. 21: 616-24 (2000).

In yet another preferred embodiment, the transgene is a marker gene that encodes a product which can be detected and used as a basis for sorting haploid cells. Preferably, the protein encoded allows for optical detection. Such a protein can be a fluorescent protein.

The term “marker gene” refers to a gene which can be used to physically separate cells expressing this marker from cells not expressing this marker. One such gene is green fluorescent protein.

The term “sort” refers to the process of creating two populations of haploid cells with one population enriched for cells containing a specific sex chromosome. This term can refer to FACS sorting, a technique which is familiar to one skilled in the art. The term may also encompass others means of creating a population of cells enriched for a specific sex chromosome such as affinity purification by a marker found on the surface of cells, or some other means of selection.

While the gene(s) described above can be expressed in the final haploid cell types produced by males and females (i.e., spermatozoa and eggs), the skilled artisan will understand that a population of these final cells enriched for cells containing a specific sex chromosome can be obtained by expressing the gene(s) in precursors to those final cells. For example, one or more transgenes can be expressed in primary spermatocytes that kill only those cells containing the transgene(s). As a result, only those cells not expressing the gene can mature into spermatozoa.

The term “X sperm” refers to a sperm or spermatozoa which includes only an X sex chromosome. Such cells may also be referred to as X-chromosome sperm or an X-chromosome-bearing sperm. Similarly, the term “Y sperm” refers to a sperm or spermatozoa which includes only a Y sex chromosome. Such cells may also be referred to as Y-chromosome sperm or an Y-chromosome-bearing sperm.

The term “enriched” means both purifying in an numerical sense and purifying in a functional sense. “Enriched” does not imply that there are no undesired cells are present, just that the relative amount of the cells of interest have been significantly increased in either a numeric or functional sense. First, by the use of the term “enriched” in referring to haploid cells in a numerical sense is meant that the desired cells constitute a significantly higher fraction (2- to 5-fold) of the total haploid cells present. This would be caused by a person by preferential reduction in the amount of the other haploid cells present.

The term “enriched” in reference to haploid cells may also mean that the specific cells desired constitute a significantly higher fraction (2- to 5-fold) of the total, functional haploid cells present. This would be caused by a person by preferential reduction in the amount of functional undesired cells. “Enriched” may also mean that one population of haploid cells is at some competitive disadvantage in comparison to another population. For example, a small decrease in fitness of, say, X chromosome-bearing sperm may dramatically reduce their ability to compete with Y chromosome-bearing sperm to fertilize an ovum.

The term “significant” is used to indicate that the level of increase is useful to the person making such an increase, and generally means an increase relative to the other of haploid cells of about at least 2-fold, more preferably at least 5- to 10-fold or even more. That is, the term is meant to cover only those situations in which a person has intervened to elevate the proportion of the desired haploid cells.

The term “functional sperm” means sperm that are capable of fertilizing ova. In preferred embodiments, a functional sperm is motile, capable of binding to ova, capable of transferring their DNA to the ova, and contain undamaged DNA. The skilled artisan will understand that not all of these characteristics are required for a sperm to function, however. For example, non-motile sperm can be directly injected into eggs to initiate fertilization.

In preferred embodiments, a transgenic animal is a mammal, most preferably an ungulate. Particularly preferred transgenic animals are selected from the group consisting of a bovid, ovid, suid, equid, caprid, and cervid.

The term “mammalian” as used herein refers to any animal of the class Mammalia. Preferably, a mammal is a placental, a monotreme and a marsupial. Most preferably, a mammalis a canid, felid, murid, leporid, ursid, mustelid, ungulate, ovid, suid, equid, bovid, caprid, cervid, and a human or non-human primate.

The term “canid” as used herein refers to any animal of the family Canidae. Preferably, a canid is a wolf, a jackal, a fox, and a domestic dog. The term “felid” as used herein refers to any animal of the family Felidae. Preferably, a felid is a lion, a tiger, a leopard, a cheetah, a cougar, and a domestic cat. The term “murid” as used herein refers to any animal of the family Muridae. Preferably, a murid is a mouse and a rat. The term “leporid” as used herein refers to any animal of the family Leporidae. Preferably, a leporid is a rabbit. The term “ursid” as used herein refers to any animal of the family Ursidae. Preferably, a ursid is a bear. The term “mustelid” as used herein refers to any animal of the family Mustelidae. Preferably, a mustelid is a weasel, a ferret, an otter, a mink, and a skunk. The term “primate” as used herein refers to any animal of the Primate order. Preferably, a prlimate is an ape, a monkey, a chimpanzee, and a lemur.

The term “ungulate” as used herein refers to any animal of the polyphyletic group formerly known as the taxon Ungulata. Preferably, an ungulate is a camel, a hippopotamus, a horse, a tapir, and an elephant. Most preferably, an ungulate is a sheep, a cow, a goat, and a pig. Especially preferred in the bovine species are Bos taurus, Bos indicus, and Bos buffaloes cows or bulls. The term “ovid” as used herein refers to any animal of the family Ovidae. Preferably, an ovid is a sheep. The term “suid” as used herein refers to any animal of the family Suidae. Preferably, a suid is a pig or a boar. The term “equid” as used herein refers to any animal of the family Equidae. Preferably, an equid is a zebra or an ass. Most preferably, an equid is a horse. The term “bovid” as used herein refers to any animal of the family Bovidae. Preferably, an bovid is an antelope, an oxen, a cow, and a bison. The term “caprid” as used herein refers to any animal of the family Caprinae. Preferably, a caprid is a goat. The term “cervid” as used herein refers to any animal of the family Cervidae. Preferably, a cervid is a deer.

In certain embodiments, this invention relates to animals in which one or more transgenes capable of being expressed in a haploid-specific manner in cells is incorporated into the genome, and hence the haploid cells, of the transgenic animal. This transgene can be under the control of a promoter region and/or an enhancer region which is capable of conferring sex chromosome-specific expression on the coupled transgene; and this transgene can also under the control of a promoter region and/or an enhancer region which only allows expression of its operably linked gene when provided specific inducing agent.

The term “inducible” refers to a promoter which is only active in the presence of specific inducing agent. Preferably the inducing agent is supplied exogenously. The inducing factor may require binding to other cellular components in order to achieve the intended result of increasing transcription. Examples of inducible promoters are well known to those skilled in the art. The exogenous inducing agent may be given to the animal producing the sperm, or it may be incubated with isolated sperm. The inducing agent may also be produced endogenously by the animal from which the enriched sperm is to be isolated.

For instance, an inducible promoter, such as the IL-8 promoter that is responsive to TNF or another cytokine, can be employed. Other examples of suitable inducible promoter systems include, but are not limited to, the metallothionine inducible promoter system, the bacterial lacZYA expression system, the tetracycline expression system, and the T7 polymerase system. Further, promoters that are selectively activated at different developmental stages (e.g., globin genes are differentially transcribed in embryos and adults) can be employed. Still other possibilities include the use of a glucocorticoid response element or a tetracycline response element.

Construction of an exogenous nucleic acid operably linked to a promoter is also well within the skill of the art (See, for example, Sambrook et al., Molecular Cloning: A Laboratory Manual, (2d ed. 1989) which is hereby incorporated by reference herein in its entirety including any figures, tables, or drawings.). With respect to the transfer and expression of exogenous nucleic acids according to the present invention, one skilled in the art is aware that different genetic signals and processing events control levels of nucleic acids and proteins/peptides in a cell, including transcription, MRNA translation, and post-transcriptional processing. Transcription of DNA into RNA requires a functional promoter.

Protein expression is dependent on the level of RNA transcription which is regulated by DNA signals. Similarly, translation of MRNA requires, at the very least, an AUG initiation codon, which is usually located within 10 to 100 nucleotides of the 5′ end of the MRNA. Sequences flanking the AUG initiator codon have been shown to influence its recognition by eukaryotic ribosomes, with conformity to a perfect Kozak consensus sequence resulting in optimal translation (see, e.g., Kozak, J. Molec. Biol., 1987, 196:947-950). Also, successful expression of an exogenous nucleic acid in a cell can require post-translational modification of a resultant protein. Thus, production of a recombinant protein can be affected by the efficiency with which DNA (or RNA) is transcribed into mRNA, the efficiency with which mRNA is translated into protein, and the ability of the cell to carry out post-translational modification. These are all factors of which one skilled in the art is aware and is capable of manipulating using standard means to achieve the desired end result.

Along these lines, to optimize protein production, preferably the transgenic nucleic acid sequence further comprises a polyadenylation site following the coding region of the transgenic nucleic acid. Also, preferably all the proper transcription signals (and translation signals, where appropriate) will be correctly arranged such that the transgenic nucleic acid sequence will be properly expressed in the cells into which it is introduced. If desired, the transgenic nucleic acid also can incorporate splice sites (i.e., splice acceptor and splice donor sites) to facilitate mRNA production. Moreover, if the transgenic nucleic acid sequence encodes a protein, which is a processed or secreted protein or functions in intracellular organelles, such as a mitochondria or the endoplasmic reticulum, preferably the transgenic nucleic acid further comprises the appropriate sequences for processing, secretion, intracellular localization, and the like. Such sequences and signals are well known to those skilled in the art.

The term “non-functional” in reference to a spermatozoa refers to cells that are no longer capable of fertilizing an ovum. This may be due to deficiencies in chromosome integrity, motility, or composition of the outer membrane.

In yet another aspect, the invention relates to methods for producing a population of haploid cells which are enriched for cells containing a specific sex chromosome, either the X or the Y, where the haploid cells are harvested from an animal comprising one or more transgenes that are capable of killing or disabling cells in cis when expressed. The transgene(s) are preferably under the control of a promoter which is only active in sperm containing a specific sex chromosome. In preferred embodiments, this promoter is active only in sperm containing a X chromosome; and this promoter is active only in sperm containing a Y chromosome. The promoter of the invention is also only active in haploid cells. The transgene then is allowed to act to kill or disable haploid cells containing the selected chromosome. Viable and/or functional haploid cells may be optionally purified away from the non-functional sperm by techniques known to those skilled in the art.

In still another aspect, the invention relates to methods for producing a population of haploid cells which are enriched for cells containing a specific sex chromosome, either the X or the Y, where the haploid cells are harvested from an animal comprising one or more transgenes which are capable of killing or disabling cells in cis when expressed, where the promoter of the invention is only active in the presence of an inducing agent. In certain preferred embodiments, this promoter is active only in haploid cells containing a X chromosome, and this promoter is active only in haploid cells containing a Y chromosome. The cells are exposed to an inducing agent, and the promoter region of the transgene(s) then acts to express the transgene(s) in cells containing one sex chromsome but not the other. The haploid cells may be exposed in vivo, either in the source animal or in the maternal host, or they may be exposed in vitro. The transgene then acts to kill or disable those haploid cells containing the selected chromosome.

In the foregoing aspects, one or more transgenes may optionally be used which do not kill or disable the haploid cells expressing the transgene(s), but rather causes the expression of a marker gene. This expressed marker may then be used to sort X-chromosome-bearing cells from Y-chromosome-bearing cells by techniques well known to those skilled in the art.

In another aspect of the invention, the invention relates to methods for producing an animal using a population of spermatozoa that is enriched for cells containing a specific sex chromosome, either the X or the Y. The offspring produced will thus be primarily of the selected sex. In preferred embodiments, if the fertilization of ova using selected sperm has been conducted in vitro, the resultant embryo is transplanted into a maternal host.

In yet another aspect, the invention relates to recombinant nucleic acids arranged and configured for performing the aspects described above, whether in vitro or in a cell or an organism. The transgenes of the instant invention are preferably comprised in the transgenic animals of the invention. The recombinant nucleic acids can alternatively contain a transcriptional initiation region functional in a cell, a sequence complementary to an RNA sequence encoding a protease polypeptide and a transcriptional termination region functional in a cell. Specific vectors and host cell combinations are discussed herein.

The present invention also relates to cells and/or organisms that contain the foregoing transgenic nucleic acid molecules incorporated into the genome, and thereby which are capable of expressing a polypeptide or other gene of interest. A cell is said to be “altered to express a desired polypeptide or other gene of interest” when the cell, through genetic manipulation, is made to produce a protein or other gene of interest which it normally does not produce or which the cell normally produces at lower levels. One skilled in the art can readily adapt procedures for introducing and expressing either genomic, cDNA, or synthetic sequences into eukaryotic cells.

A nucleic acid molecule, such as DNA, is said to be “capable of expressing” a polypeptide or other gene of interest if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region and other 5′-non-coding sequences involved with initiation of transcription and translation, such as the TATA box, capping sequence, CAAT sequence, and the like.

Two DNA sequences (such as a promoter region sequence and a sequence encoding the gene of interest) are said to be operably linked if the nature of the linkage between the two DNA sequences does not (1) result in the introduction of a frame-shift mutation, (2) interfere with the ability of the promoter region sequence to direct the transcription of a gene sequence encoding the gene of interest, or (3) interfere with the ability of the gene sequence of the gene of interest to be transcribed by the promoter region sequence. Thus, a promoter region would be operably linked to a DNA sequence if the promoter were capable of effecting transcription of that DNA sequence. Thus, to express a gene encoding the gene of interest, transcriptional and translational signals recognized by an appropriate host are necessary.

The present invention encompasses the expression of a gene encoding the gene of interest (or a functional derivative thereof) in eukaryotic cells.

The selection of control sequences, expression vectors, transformation methods, and the like, are dependent on the type of host cell used to express the gene, and their selection is well within the skill of the artisan.

As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

The term “vector” relates to a single or double-stranded circular nucleic acid molecule that can be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a protease can be inserted into a vector by cutting the vector with restriction enzymes and ligating the two pieces together. Preferred vectors are those designed for performing “gene targeting” procedures. See, e.g., U.S. Pat. Nos. 6,090,554, 6,069,010, 5,792,663, and 5,789,215, each of which is hereby incorporated by reference in its entirety, including all tables, figures, and claims.

The term “transfecting” defines a number of methods to insert a nucleic acid vector or other nucleic acid molecules into a cellular organism. These methods involve a variety of techniques, such as treating the cells with high concentrations of salt, an electric field, detergent, or DMSO to render the outer membrane or wall of the cells permeable to nucleic acid molecules of interest or use of various viral transduction strategies.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of the transgenes of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).

Translation of eukaryotic mRNA is initiated at the codon which encodes the first methionine. For this reason, it is preferable to ensure that the linkage between a eukaryotic promoter and a DNA sequence which encodes the gene of interest (or a functional derivative thereof) does not contain any intervening codons which are capable of encoding a methionine (i.e., AUG). The presence of such codons results either in the formation of a fusion protein (if the AUG codon is in the same reading frame as the protease of the invention coding sequence) or a frame-shift mutation (if the AUG codon is not in the same reading frame as the protease of the invention coding sequence).

A nucleic acid molecule encoding the gene of interest and an operably linked promoter may be introduced into a recipient host cell either as a nonreplicating DNA or RNA molecule, which may either be a linear molecule or, more preferably, a closed covalent circular molecule. Permanent expression will occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of MRNA. These elements may include splice signals, as well as transcription promoters, enhancers, and termination signals. cDNA expression vectors incorporating such elements include those described by Okayama (Mol. Cell. Biol. 3:280-289, 1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of the gene of interest, or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

### BRIEF DESCRIPTION OF THE FIGURES

FIGS. 1 shows, in schematic form, spermatogenesis, i.e., the production of haploid cells from diploid precursors that occurs in male animals.

FIG. 2 shows, in schematic form, an exemplary procedure for producing a transgenic animal of the invention.

### DETAILED DESCRIPTION OF THE INVENTION

The present invention describes materials and methods for producing semen that is enriched for active sperm containing either the X chromosome or the Y chromosome, by producing transgenic animals that express one or more genes in a sex chromosome-specific and/or haploid-specific manner. As discussed above, the ability to pre-select the sex of an offspring is particularly advantageous in the agricultural industry. By allowing for the selection of a specific population of haploid cells, the materials and methods described herein can facilitate this sex pre-selection.

I. Transgenic Cells and Animals

A. General Methods

Materials and methods readily available to a person of ordinary skill in the art can be applied to produce transgenic cells and animals. See, e.g., EPO 264 166, entitled “Transgenic Animals Secreting Desired Proteins Into Milk”; WO 94/19935, entitled “Isolation of Components of Interest From Milk”; WO 93/22432, entitled “Method for Identifying Transgenic Pre-implantation Embryos”; WO 95/17085, entitled “Transgenic Production of Antibodies in Milk;” Hammer et al., 1985, Nature 315: 680-685; Miller et al., 1986, J. Endocrinology 120: 481-488; Williams et al., 1992, J. Ani. Sci. 70: 2207-2111; Piedrahita et al., 1998, Biol. Reprod. 58: 1321-1329; Piedrahita et al., 1997, J. Reprod. Fert. (suppl.) 52: 245-254; and Nottle et al, 1997, J. Reprod. Fert. (suppl.) 52: 245-254, each of which is incorporated herein by reference in its entirety including all figures, drawings and tables.

Methods for generating transgenic cells typically include the steps of (1) assembling a suitable DNA construct useful for inserting a specific DNA sequence into the nuclear genome of a cell; (2) transfecting the DNA construct into the cells; (3) allowing random insertion and/or homologous recombination to occur. The modification resulting from this process may be the insertion of a suitable DNA construct(s) into the target genome; deletion of DNA from the target genome; and/or mutation of the target genome.

DNA constructs can comprise a gene of interest as well as a variety of elements including regulatory promoters, insulators, enhancers, and repressors as well as elements for ribosomal binding to the RNA transcribed from the DNA construct. DNA constructs can also encode ribozymes and anti-sense DNA and/or RNA, identified previously herein. These examples are well known to a person of ordinary skill in the art and are not meant to be limiting.

Due to the effective recombinant DNA techniques available in conjunction with DNA sequences for regulatory elements and genes readily available in data bases and the commercial sector, a person of ordinary skill in the art can readily generate a DNA construct appropriate for establishing transgenic cells using the materials and methods described herein.

Preferred vectors for use in the present invention are gene targeting vectors, in order to mediate insertion of a gene of interest by homologous recombination with a site in the host genome. Such vectors typically include four major elements. A promoter, is linked to, and drives, the expression of a gene. An Y or X chromosome specific DNA sequence is linked to the promoter/gene elements. The Y or X chromosome specific sequence is to be used as homologous arms for targeting the vector to the Y or X chromosome, respectively. Finally, a selection marker, such as the neomycin-resistance gene, neo (Southern, P. J. & Berg, P. (1982) J Mol Appl Genet 1: 327-341) is typically included.

Preferred elements of the vectors which may be obtained and incorporated into the targeting vectors include novel sequence of both the bovine (Lee et al (1987) Biol Chem Hoppe Seyler 368: 131-135; Krawetz et al., (1988) J Biol Clern 263: 321-326) and porcine (Maier et al., (1988) Nucleic Acids Res 16: 11826) protamine promoters. In addition, a preferred toxic gene, a dominant negative mutant of hamster BiP protein, plus wild-type hamster BiP protein, to disrupt proper protein folding in X- or Y-bearing sperm have been disclosed (Hendershot et al., (1996) Proc Natl Acad Sci U S A 93: 5269-5274; Morris et al., (1997) J Biol Chem 272: 4327-4334). Suitable bovine and porcine Y chromosome specific sequences 3′ of the SRY gene (Hacker et al., (1995) Development 121: 1603-1614) to be used as homologous arms for gene targeting have also been disclosed.

As described below, complete insertion vectors containing the promoter, gene sequence, selectable marker, and a homologous arm have been constructed. A schematic of such a vector is provided below. The vector can be linearized by cutting with a restriction enzyme that bisects the homologous arm prior to transfection to provide a mature insertion vector.

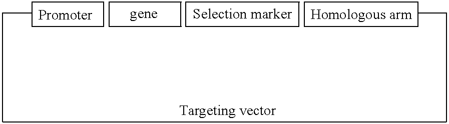

Transfection techniques are well known to a person of ordinary skill in the art and materials and methods for carrying out transfection of DNA constructs into cells are commercially available. For example, materials that can be used to transfect cells with DNA constructs are lipophillic compounds such as Lipofectin™, activated polycationic dendrimers such as Superfect™, LipoTAXI™, and CLONfectin™. Particular lipophillic compounds can be induced to form liposomes for mediating transfection of the DNA construct into the cells. In addition, cationic based transfection agents that are known in the art can be utilized to transfect cells with nucleic acid molecules (e.g., calcium phosphate precipitation). Also, electroporation techniques known in the art can be utilized to translocated nucleic acid molecules into cells. Furthermore, particle bombardment techniques known in the art can be utilized to introduce exogenous DNA into cells. Target sequences from a DNA construct can be inserted into specific regions of the nuclear genome by rational design of the DNA construct. These design techniques and methods are well known to a person of ordinary skill in the art. See, U.S. Pat. No. 5,633,067, “Method of Producing a Transgenic Bovine or Transgenic Bovine Embryo,” DeBoer et al., issued May 27, 1997; U.S. Pat. No. 5,612,205, “Homologous Recombination in Mammalian Cells,” Kay et al., issued Mar. 18, 1997; and PCT publication WO 93/22432, “Method for Identifying Transgenic Pre-Implantation Embryos,” each of which is incorporated herein by reference in its entirety, including all figures, drawings, and tables. Once the desired DNA sequence is inserted into the nuclear genome of a cell, the location of the insertion region as well as the frequency with which the desired DNA sequence has inserted into the nuclear genome can be identified by methods well known to those skilled in the art.

B. Haploid-Specific Expression

In a preferred embodiment, the protamine promoter can be used to establish haploid-specific and/or tissue-specific gene expression. Protamine is a small, basic protein that binds to DNA during the condensation and compaction of the sperm head. Protamine is expressed exclusively in testis, and it is expressed at the haploid stage in round spermatids following the completion of meiosis. Lee et al., 1987, Biol. Chem. Hoppe Seyler 970: 807-1 1. Regulatory sequences for this gene have been found in about 10 species, including bovines. Krawetz et al., 1988, J. Biol. Chem. 263: 321-326; Queralt and Olivia, 1993, Gene 133: 197-204.

C. Expression of a Gene Product In Cis

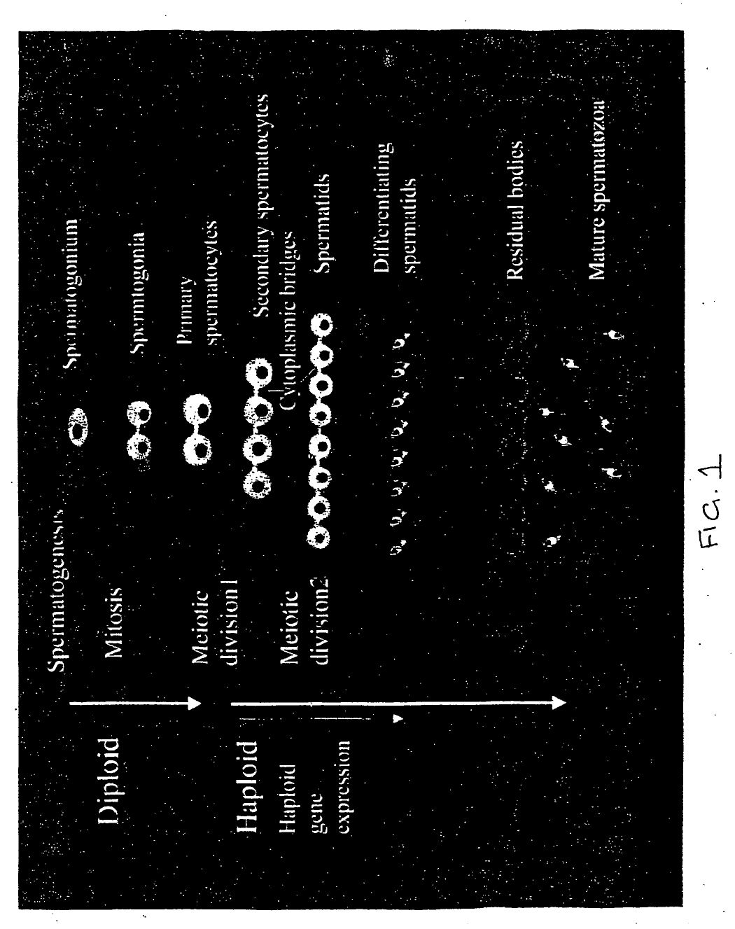

During spermatogenesis, haploid cells at certain stages are joined by “cytoplasmic bridges” that allow sharing of soluble cell contents between adjacent cells. See, e.g., FIG. 1. Thus, if a transgene is selected that produces a freely soluble expression product, and the construct chosen allows expression at the stage when these bridges are present, the expression product may kill cells containing both sex chromosomes. Therefore, it may be important to select a gene product that produces its effects only in cis. Such a gene product preferably exhibits the following characteristics: the ability to exert its effects in a dominant fashion (i.e., expression of the transgene alone creates the effect, even against an otherwise wild type expression background); the ability to remain anchored to the matrix of the cell in which it is produced; and participation in an essential function, so that expression causes death or disablement of the cell.

In this regard, a preferred gene, the expression of which can be driven by the protamine promoter in a haploid-specific fashion, is the immunoglobulin heavy chain binding protein (BiP). BiP is a HSP 70 molecular chaperone. A series of point mutations in a hamster BiP sequence has been shown to inhibit the BiP ATPase activity, resulting in a dominant negative mutant exhibiting disrupted endoplasmic reticulum (ER) function. See, e.g., Hendershot et al., Proc. Natl. Acad. Sci. USA 93: 5269-74 (1996). Furthermore, this dominant negative effect can cross species; i.e., hamster BiP mutants can disrupt ER function in bovines for example.

Expression of such a mutant in spermatids can disrupt the normal development of spermatozoa. By using gene targeting methods targeted at a Y chromosome-specific or X chromosome-specific intronic sequence, BiP expression can be made both haploid-specific and sex chromosome-specific.

II. Nuclear Transfer

In preferred embodiments, once a transgene(s) is (are) inserted into the nuclear genome of the totipotent cell, that cell can be used as a nuclear donor for cloning a transgenic animal.

Nuclear transfer (NT) techniques are well known to a person of ordinary skill in the art. See, e.g., U.S. Pat. No. 4,664,097, “Nuclear Transplantation in the Mammalian Embryo by Microsurgery and Cell Fusion,” issued May 12, 1987, McGrath & Solter; U.S. Pat. No. 4,994,384 (Prather et al.); U.S. Pat. No. 5,057,420 (Massey et al.); U.S. Pat. No. 6,107,543; U.S. Pat. No. 6,011,197; Proc. Nat'l. Acad. Sci. USA 96: 14984-14989 (1999); Nature Genetics 22: 127-128 (1999); Cell & Dev. Diol 10: 253-258 (1999); Nature Biotechnology 17: 456-461 (1999); Science 289: 1188-1190 (2000); Nature Biotechnol. 18: 1055-1059 (2000); and Nature 407: 86-90 (2000); each of which is incorporated herein by reference in its entirety, including all figures, tables, and drawings. Exemplary embodiments define a NT technique that provide for efficient production of totipotent mammalian embryos.

A. Nuclear Donors

For NT techniques, a donor cell may be separated from a growing cell mass, isolated from a primary cell culture, or isolated from a cell line. The entire cell may be placed in the perivitelline space of a recipient oocyte or may be directly injected into the recipient oocyte by aspirating the nuclear donor into a needle, placing the needle into the recipient oocyte, releasing the nuclear donor and removing the needle without significantly disrupting the plasma membrane of the oocyte. Also, a nucleus (e.g., karyoplast) may be isolated from a nuclear donor and placed into the perivitelline space of a recipient oocyte or may be injected directly into a recipient oocyte, for example.

B. Recipient Cells

A recipient cell is typically an oocyte with a portion of its ooplasm removed, where the removed ooplasm comprises the oocyte nucleus. Enucleation techniques are well known to a person of ordinary skill in the art. See e.g., Nagashima et al., 1997, Mol. Reprod. Dev. 48: 339-343; Nagashima et al., 1992, J. Reprod. Dev. 38: 37-78; Prather et al., 1989, Biol. Reprod. 41: 414-418; Prather et al., 1990, J. Exp. Zool. 255: 355-358; Saito et al., 1992, Assis. Reprod. Tech. Andro. 259: 257-266; and Terlouw et al., 1992, Theriogenology 37: 309, each of which is incorporated herein by reference in its entirety including all figures, tables, and drawings. Cells other than oocytes can also be successfully used as recipient cells. See, e.g., Polejaeva et al., Nature 407(6800): 86-90 (2000).

Oocytes can be isolated from either oviducts and/or ovaries of live animals by oviductal recovery procedures or transvaginal oocyte recovery procedures well known in the art and described herein. Furthermore, oocytes can be isolated from deceased animals. For example, ovaries can be obtained from abattoirs and oocytes can be aspirated from these ovaries. The oocytes can also be isolated from the ovaries of a recently sacrificed animal or when the ovary has been frozen and/or thawed.

Oocytes can be matured in a variety of media well known to a person of ordinary skill in the art. One example of such a medium suitable for maturing oocytes is depicted in an exemplary embodiment described hereafter. Oocytes can be successfully matured in this type of medium within an environment comprising 5% CO₂ at 39° C. Oocytes may be cryopreserved and then thawed before placing the oocytes in maturation medium. Cryopreservation procedures for cells and embryos are well known in the art as discussed herein.

Components of an oocyte maturation medium can include molecules that arrest oocyte maturation. Examples of such components are 6-dimethylaminopurine (DMAP) and isobutylmethylxanthine (IBMX). IBMX has been reported to reversibly arrest oocytes, but the efficiencies of arrest maintenance are quite low. See, e.g., Rose-Hellkant and Bavister, 1996, Mol. Reprod. Develop. 44: 241-249. However, oocytes may be arrested at the germinal vesicle stage with a relatively high efficiency by incubating oocytes at 31° C. in an effective concentration of IBMX. Preferably, oocytes are incubated the entire time that oocytes are collected. Concentrations of IBMX suitable for arresting oocyte maturation are 0.01 mM to 20 mM IBMX, preferably 0.05 mM to 10 mM IBMX, and more preferably about 0.1 mM IBMX to about 0.5 mM IBMX, and most preferably 0.1 mM IBMX to 0.5 mM IBMX. In certain embodiments, oocytes can be matured in a culture environment having a low oxygen concentration, such as 5% O₂, 5-10% CO₂, and 85-90% N₂.

A nuclear donor cell and a recipient oocyte can arise from the same species or different species. For example, a totipotent porcine cell can be inserted into a porcine enucleated oocyte. Alternatively, a totipotent wild boar cell can be inserted into a domesticated porcine oocyte. Any nuclear donor/recipient oocyte combinations are envisioned by the invention. Preferably the nuclear donor and recipient oocyte from the same specie. Cross-species NT techniques can be utilized to produce cloned animals that are endangered or extinct.

Oocytes can be activated by electrical and/or non-electrical means before, during, and/or after a nuclear donor is introduced to recipient oocyte. For example, an oocyte can be placed in a medium containing one or more components suitable for non-electrical activation prior to fusion with a nuclear donor. Also, a cybrid can be placed in a medium containing one or more components suitable for non-electrical activation. Activation processes are discussed in greater detail hereafter.

C. Injection/Fusion

A nuclear donor can be translocated into an oocyte using a variety of materials and methods that are well known to a person of ordinary skill in the art. In one example, a nuclear donor may be directly injected into a recipient oocyte. This direct injection can be accomplished by gently pulling a nuclear donor into a needle, piercing a recipient oocyte with that needle, releasing the nuclear donor into the oocyte, and removing the needle from the oocyte without significantly disrupting its membrane. Appropriate needles can be fashioned from glass capillary tubes, as defined in the art and specifically by publications incorporated herein by reference.

In another example, at least a portion of plasma membrane from a nuclear donor and recipient oocyte can be fused together by utilizing techniques well known to a person of ordinary skill in the art. See, Willadsen, 1986, Nature 320:63-65, hereby incorporated herein by reference in its entirety including all figures, tables, and drawings. Typically, lipid membranes can be fused together by electrical and chemical means, as defined previously and in other publications incorporated herein by reference.

Examples of non-electrical means of cell fusion involve incubating cybrids in solutions comprising polyethylene glycol (PEG), and/or Sendai virus. PEG molecules of a wide range of molecular weight can be utilized for cell fusion.

Processes for fusion that are not explicitly discussed herein can be determined without undue experimentation. For example, modifications to cell fusion techniques can be monitored for their efficiency by viewing the degree of cell fusion under a microscope. The resulting embryo can then be cloned and identified as a totipotent embryo by the same methods as those previously described herein for identifying totipotent cells, which can include tests for selectable markers and/or tests for developing an animal.

D. Activation

Methods of activating oocytes and cybrids are known to those of ordinary skill in the art. See, U.S. Pat. No. 5,496,720, “Parthenogenic Oocyte Activation,” Susko-Parrish et al., issued on Mar. 5, 1996, hereby incorporated by reference herein in its entirety including all figures, tables, and drawings.

Both electrical and non-electrical processes can be used for activating cells (e.g., oocytes and cybrids). Although use of a non-electrical means for activation is not always necessary, non-electrical activation can enhance the developmental potential of cybrids, particularly when young oocytes are utilized as recipients.

Examples of electrical techniques for activating cells are well known in the art. See, WO 98/16630, published on Apr. 23, 1998, Piedraheidra and Blazer, hereby incorporated herein in its entirety including all figures, tables, and drawings, and U.S. Pat. Nos. 4,994,384 and 5,057,420. Non-electrical means for activating cells can include any method known in the art that increases the probability of cell division. Examples of non-electrical means for activating a nuclear donor and/or recipient can be accomplished by introducing cells to ethanol; inositol trisphosphate (IP₃); Ca²⁺ ionophore and protein kinase inhibitors such as 6-dimethylaminopurine; temperature change; protein synthesis inhibitors (e.g., cycloheximide); phorbol esters such as phorbol 12-myristate 13-acetate (PMA); mechanical techniques, thapsigargin, and sperm factors. Sperm factors can include any component of a sperm that enhance the probability for cell division. Other non-electrical methods for activation include subjecting the cell or cells to cold shock and/or mechanical stress.

Examples of preferred protein kinase inhibitors are protein kinase A, G, and C inhibitors such as 6-dimethylaminopurine (DMAP), staurosporin, 2-aminopurine, sphingosine. Tyrosine kinase inhibitors may also be utilized to activate cells.

Activation materials and methods that are not explicitly discussed herein can be identified by modifying the specified conditions defined in the exemplary protocols described hereafter and in U.S. Pat. No. 5,496,720.

F. Manipulation of Embryos Resulting from Nuclear Transfer

An embryo resulting from a NT process can be manipulated in a variety of manners. The invention relates to cloned embryos that arise from at least one NT. Exemplary embodiments of the invention demonstrate that two or more NT procedures may enhance the efficiency for the production of totipotent embryos. Exemplary embodiments indicate that incorporating two or more NT procedures into methods for producing cloned totipotent embryos may enhance placental development. In addition, increasing the number of NT cycles involved in a process for producing totipotent embryos may represent a necessary factor for converting non-totipotent cells into totipotent cells. An effect of incorporating two or more NT cycles upon totipotency of resulting embryos is a surprising result, which was not previously identified or explored in the art.

Incorporating two or more NT cycles into methods for cloned totipotent embryos can provide further advantages. Incorporating multiple NT procedures into methods for establishing cloned totipotent embryos provides a method for multiplying the number of cloned totipotent embryos.

When multiple NT procedures are utilized for the formation of a cloned totipotent embryo, oocytes that have been matured for any period of time can be utilized as recipients in the first, second or subsequent NT procedures. Additionally, one or more of the NT cycles may be preceded, followed, and/or carried out simultaneously with an activation step. As defined previously herein, an activation step may be accomplished by electrical and/or non-electrical means as defined herein. Exemplified embodiments described hereafter describe NT techniques that incorporate an activation step after one NT cycle. However, an activation step may also be carried out at the same time as a NT cycle (e.g., simultaneously with the NT cycle) and/or an activation step may be carried out prior to a NT cycle. Cloned totipotent embryos resulting from a NT cycle can be (1) disaggregated or (2) allowed to develop further.

If embryos are disaggregated, disaggregated embryonic derived cells can be utilized to establish cultured cells. Any type of embryonic cell can be utilized to establish cultured cells. These cultured cells are sometimes referred to as embryonic stem cells or embryonic stem-like cells in the scientific literature. The embryonic stem cells can be derived from early embryos, morulae, and blastocyst stage embryos. Multiple methods are known to a person of ordinary skill in the art for producing cultured embryonic cells. These methods are enumerated in specific references previously incorporated by reference herein.

If embryos are allowed to develop into a fetus in utero, cells isolated from that developing fetus can be utilized to establish cultured cells. In preferred embodiments, primordial germ cells, genital ridge cells, and fetal fibroblast cells can be isolated from such a fetus. Cultured cells having a particular morphology that is described herein can be referred to as embryonic germ cells (EG cells). These cultured cells can be established by utilizing culture methods well known to a person of ordinary skill in the art. Such methods are enumerated in publications previously incorporated herein by reference and are discussed herein. In particularly preferred embodiments, Streptomyces griseus protease can be used to remove unwanted cells from theembryonic germ cell culture.

Cloned totipotent embryos resulting from NT can also be manipulated by cryopreserving and/or thawing the embryos. See, e.g., Nagashima et al., 1989, Japanese J. Anim. Reprod. 35: 130-134 and Feng et al., 1991, T7Theriogenology 35: 199, each of which is incorporated herein by reference in its entirety including all tables, figures, and drawings. Other embryo manipulation methods include in vitro culture processes; performing embryo transfer into a maternal recipient; disaggregating blastomeres for NT processes; disaggregating blastomeres or inner cell mass cells for establishing cell lines for use in NT procedures; embryo splitting procedures; embryo aggregating procedures; embryo sexing procedures; and embryo biopsying procedures. The exemplary manipulation procedures are not meant to be limiting and the invention relates to any embryo manipulation procedure known to a person of ordinary skill in the art.

III. Development of Cloned Embryos

A. Culture of Embryos In Vitro

Cloning procedures discussed herein provide an advantage of culturing cells and embryos in vitro prior to implantation into a recipient female. Methods for culturing embryos in vitro are well known to those skilled in the art. See, e.g., Nagashima et al., 1997, Mol. Reprod. Dev. 48: 339-343; Petters & Wells, 1993, J. Reprod. Fert. (Suppl) 48: 61-73; Reed et al., 1992, Theriogenology 37: 95-109; and Dobrinsky et al., 1996, Biol. Reprod. 55: 1069-1074, each of which is incorporated herein by reference in its entirety, including all figures, tables, and drawings. In addition, exemplary embodiments for media suitable for culturing cloned embryos in vitro are described hereafter. Feeder cell layers may or may not be utilized for culturing cloned embryos in vitro. Feeder cells are described previously and in exemplary embodiments hereafter.

B. Development of Embryos In Utero

Cloned embryos can be cultured in an artificial or natural uterine environment after NT procedures and embryo in vitro culture processes. Examples of artificial development environments are being developed and some are known to those skilled in the art. Components of the artificial environment can be modified, for example, by altering the amount of a component or components and by monitoring the growth rate of an embryo.

Methods for implanting embryos into the uterus of an animal are also well known in the art, as discussed previously. Preferably, the developmental stage of the embryo(s) is correlated with the estrus cycle of the animal.

Embryos from one species can be placed into the uterine environment of an animal from another species. For example it has been shown in the art that bovine embryos can develop in the oviducts of sheep. Stice & Keefer, 1993, “Multiple generational bovine embryo cloning,” Biology of Reproduction 48: 715-719. The invention relates to any combination of a porcine embryo in any other ungulate uterine environment. A cross-species in utero development regime can allow for efficient production of cloned animals of an endangered species. For example, a wild boar embryo can develop in the uterus of a domestic porcine sow.

Once an embryo is placed into the uterus of a recipient female, the embryo can develop to term. Alternatively, an embryo can be allowed to develop in the uterus and then can be removed at a chosen time. Surgical methods are well known in the art for removing fetuses from uteri before they are born.

### EXAMPLES

The examples below are not limiting and are merely representative of various aspects and features of the present invention.

### Example 1

### Targeting Vectors

Preferred targeting vectors include four major elements. A promoter, preferably the protamine gene promoter, is linked to, and drives, the expression of a gene, preferably the hamster BiP protein, to disrupt sperm development. Both wild-type and mutant hamster BiP genes may be used to prepare vectors. The third element of the vectors is a Y or X chromosome specific DNA sequence which is linked to the promoter/gene elements. The Y or X chromosome specific sequence is to be used as homologous arms for targeting the vector to the Y or X chromosome, respectively. The fourth element of the vectors is a selection marker, such as the neomycin-resistance gene, neo (Southern, P. J. & Berg, P. (1982) J Mol Appl Genet 1: 327-341).

Preferred elements of the vectors which may be obtained and incorporated into the targeting vectors include novel sequence of both the bovine (Lee et al (1987) Biol Chem Hoppe Seyler 368: 131-135; Krawetz et al., (1988) J Biol Chem 263: 321-326) and porcine (Maier et al., (1988) Nucleic Acids Res 16: 11826) protamine promoters. In addition, a preferred toxic gene, a dominant negative mutant of hamster BiP protein, plus wild-type hamster BiP protein, to disrupt proper protein folding in X- or Y-bearing sperm have been disclosed (Hendershot et al., (1996) Proc Natl Acad Sci U S A 93: 5269-5274; Morris et al., (1997) J Biol Chem 272: 4327-4334). Suitable bovine and porcine Y chromosome specific sequences 3′ of the SRY gene (Hacker et al., (1995) Development 121: 1603-1614) to be used as homologous arms for gene targeting have also been disclosed.

As described below, complete insertion vectors containing the bovine protamine promoter, mutant or wild-type BiP cDNA, the neomycin-resistent marker neo, and a homologous arm with bovine Y chromosome specific sequence have been constructed. The backbone for the insertion vector was pGT-N29 (New England Biolabs #N3729S). Preferred insertion sites are Xho I and/or Bsi WI in the vector. A diagram of the constructs is shown below.

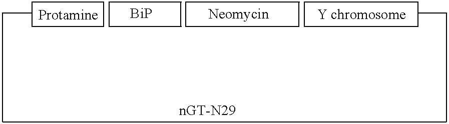

Promoter Sequences

In 1988, Krawetz et al. published a bovine protamine 1 gene cDNA sequences with 597 bp of 5′ flanking region. Krawetz et al., (1988) J Biol Chem 263: 321-326. In order to obtain a more complete promoter sequence, PCR of bovine genomic DNA was performed using forward (nt 615-640) and reverse (nt 1003-1028) primers from the published sequences. A fragment of genomic DNA containing the cDNA and the intron of protamine 1 was obtained. The fragment was used as a probe to isolated a cosmid clone from bovine genomic library (Genome System Bovine Cosmid Library, clone address 180P13).

According to the data obtained, it was determined that nucleotide 1 to 207 of the published sequences of bovine protamine 1 are actually protamine 2 sequences which were mistakenly assigned to the protamine 1 sequence. Thus, the actual sequence of protamine 1 begins from nucleotide 208 of the published sequence, and contains only 390 bp of the 5′ flanking region. In addition, a ˜1 kb sequence which is located further upstream of the protamine 1 gene was obtained.

TABLE 1

Published bovine protamine 1 gene sequence
(5′ to 3′) (SEQ ID NO: 1).

| | |
|---|---|
| 1 | TCGAAACCAG GGGACAAAAC CTCTGAAGAT GAGGGCCAGC |
| | CTCCTTGTCT GGATCCAAGC |
| 61 | CCTCACACCC TGCCCCTCCC CCAGCTCCTC GGGGTTCCTG |
| | AAGCTTCCCT GCTGCCTTTG |
| 121 | CAGCCACTGC TGTGGCCTCT CGGGGGGCTG GGATGGGGGC |
| | TTATCTGTCC ACAGGGTTAT |
| 181 | CTTATGCTCA CTCTGTGCCA G [symbol] CTC CTTTACAGAG |
| | GAGGAGGCAT GGAGACTTGG |
| 241 | ACGTCATAGC TGGGTTCGGG CTGCTCATGG GGTCTTGGAC |
| | CAGCTTGGCA GGAACTGTCA |
| 301 | TGACTCCTCT ACCTCCCCCC CCTCCCCACT GCATGATGTG |
| | ATGTGGTCAA ATTTATATGC |
| 361 | ATTAATGACC TGGGGGGTCA TTAATTAATG TGGAGGGGCC |
| | CCACCCCCCC CCACATCACA |
| 421 | GCCCCACCCC TGCACATCAC AGCCCCGCCC TCCCTCACCA |
| | AGCACCTCCC ACATGCCCAT |
| 481 | ATATGGGCAT GATTTGGGCA GCTCTGACCC TGGTCTGTGA |
| | GGTCTGGGTC TCTGTGACCT |
| 541 | CACAATGACC AGGGCCCTGC CCGGGTCTAT ATAAGAGGCC |
| | AGGAAGTCGG CCCCTGTC*AC |
| 601 | AGCCCACAAA TTCCACCTGC TCACAGGTTG GCTGGCTCAA |
| | CCAAGGCGGT ATCCCCTGCT |
| 661 | CTGAGCATCC AGGCCGAATC CACCCAGCAC CATGGCCAGA |
| | TACCGATGCT GCCTCACCCA |
| 721 | TAGCGGGAGC AGATGCCGCC GCCGCCGCCG AAGAAGATGT |
| | CGCAGACGAA GGAGGCGCTT |
| 781 | TGGTCGGAGG CGCAGGAGGA [symbol] [symbol] |
| | [symbol] [symbol] |
| 841 | [symbol] [symbol] [symbol] [symbol] |
| | [symbol] [symbol] |
| 901 | GCAGTGTGCT GCCGTCGCTA CACCGTCATA AGGTGTACAA |
| | GACAGTAACC ACACAGTAGC |
| 961 | AAGACCACCG CACTCCTGCC TGAAAGGTCA CCAGCCTTCA |
| | AGACCCTCTT GCCACATCTT |
| 1021 | GAACATGCCA CCATTTCAAT GACATGAACA GGAGCCTGCT |
| | AACGAACAAT GCCACCTGTC |
| 1081 | AATAAATGTT GAAAGACATC ATTCCACTCT TTGACTCTTT |
| | GCTTTGAGGG ACTCTAGGCG |
| 1141 | GGGTGGGGGG GGGGGGGAAG GAGGGGGTTG GGGATGCTGG |
| | ATCTTGTTCC AAACTCAACT |
| 1201 | ACTCCCGAGT CACAAACCAA ACCTGCCTCC CAGCCCCTAG |
| | TCCTTTACAG ACCCCTTTCC |
| 1261 | AGCGGGGACG GGAGCTGTGC TGGTTGATGA ACACATCCCT |
| | CCCCAGTTCT GTGCTCAGTG |
| 1321 | GCTTTCTACT GACAGCTCGA |

The EcoRI site at nt 202-207 is italicized and underlined. The star indicates the transcription start site and the atg start codon is underlined. The italicized and bold sequence is the intron 1 region of protamine 1.

The sequence obtained in the present invention are provided below in Table 2. As obtained and presented, this sequence is reversed, and is complementary to the sequence shown in Table 1. The first 48 nt match with nt 249-202 of the published sequences (thus the first three nucleotides (CTA) in this sequence are complementary to the three nucleotides beginning at nt 247 of Table l(reading backward, GAT)).

TABLE 2

Sequence of the 5′ flanking region of bovine protamine 1 (3′ to 5′)
(SEQ ID NO: 2)

CTATGACGTCCAAGTCTCCATGCCTCCTCCTCTGTAAAGGAGGAATTC

[symbol]

[symbol]

[symbol]

[symbol]

[symbol]

[symbol]

[symbol]

[symbol]

[symbol]

[symbol]

[symbol]

[symbol]

[symbol]

TGCAAGAAATC
AAAGGGAGGCCGAGGGGGACGGAGCANGAGAGTGCGGGGGAAGGGTGGGCACAACAGATAAGGAAGGTAG
CAATTAGAATTTGAAATCGTTACTCATAGCAGGAAACCAAAATAAGTGTCTTTGGCATGTGNNGGNGGTT
TAGTCACCAAGTTGTGTCCAACTTCTTGCAACCCCATGGACTGTAGCCCGCCAGCTCCNTCTGTCCATGG
GATTCTCCAGGCAAGAATACTGGAATGGGTTGCTATTTCCTTCTCCTGGGGATCTTCCCAACCT -5′

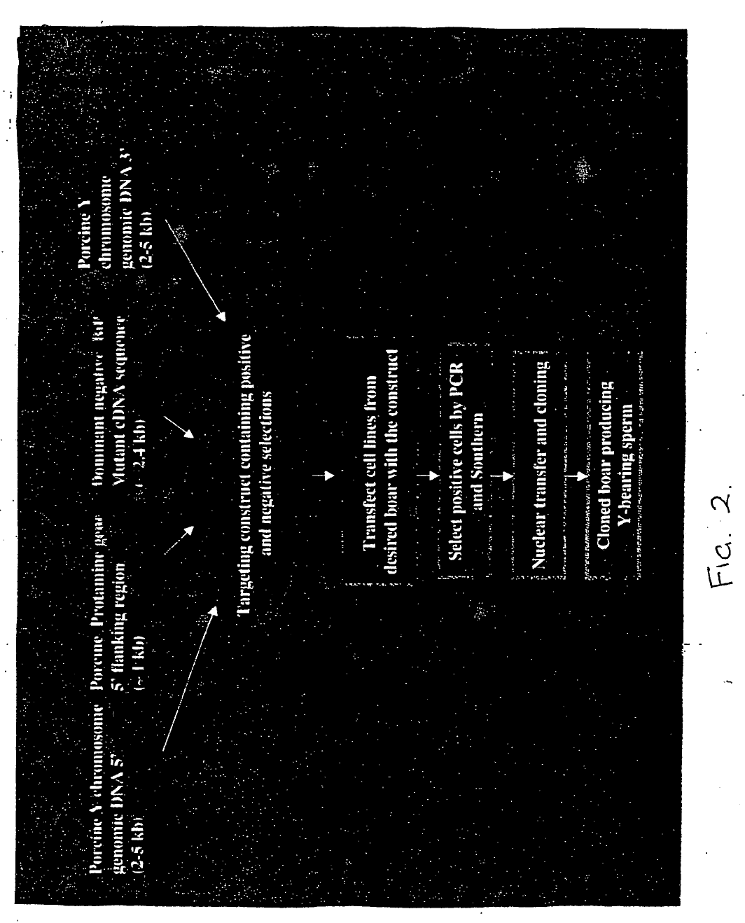

A preferred promoter sequence used in the present invention is shown below in Table 3. This promoter sequence is shown in the same orientation as that of Table 1, and is thus the reverse complement of the sequence in Table 2. The sequences contain nt 202-nt 690 (before the atg start codon) of the published bovine protamine 1 sequence (shown in italics) and 852 bp of sequence obtained in the present invention (shown in bold, also shown in FIG. 2 in italics, including the underlined region).

TABLE 3

Preferred bovine promoter for use in the bovine targeting construct
(5′ to 3′) (SEQ ID NO: 3)

5′CCTTCCAAGCAACTTTCAAGCCTAAGACTTTTTTTTTTTTTTAACCCAATTGAGGGTAAAAATTCACA
TCGTGGTAAAATCCAGCATACTAAAAAAGTGAAAATTCAGTGTCAATGAAAACATTCACCATGTTGGGTA
ACTGTCATCACTATCTGGGTTCAAAACATATTTATTCACACCAAAAGGAGCCCCTGCACCGCTTATGGAG
CAGCCGCTCCCATTTCACCCGCTTCTGGGCAACCACCAACCCATTCCTGCGCCGGTGGCCTTCCCTCAGT
GGCTGTTTCACTACTGGAATCACACACGTGCTGCCTCAGGACTTGGCAGCAGCTCATCCCTCTTCCCAGA
TGCTCTTCCCTCCTCCCACTCCAATCATCTTCCTTAGGAGGCCTTCCTGGGGCCTCTCCTGAGGTCAAGT
CCTCCAGCATAACTTTCTCATAAAAGCCCAGAGTTTCTCTCTGAGGCATTTTTTTCACAAGTGCTCTGCA
TGGTAATTTCATTGGGTTTCCCTGGTGGCTCAGACGGTAAAGAATCTGCCTGTGATGCAAGAGACCCGGG
TTCGATCTCTGGGCTGGGAAGATCCCCTGGAGGAAGCATGGGCAACCTACTCCAATATTCTCGGCTGGAG
CACTCCATGGACGGAGGAGTCTGGTGGGCTACATATAGTCTGTGGGGTTACAGTTCATGTTCTCTACATT
AAGAATGTAAAGTTTATTAGGCAGGAACATGTCTGACTTGTTTACCAGTATTTGCCAGGGCCTCGCCAGA
ACCTGGCATACACTGTAAACTGACTGAGTGGATGAGCTAGTGAGTGTCCTTACAAAGAAAAAGTCACCTC
ATTCCTCTTTACAGAATTCCTCCTTTACAGAGGAGGAGGCATGGAGACTTGGGCCGTCATAGCTGGGTTC
GGGCTGCTCATGGGGTCTTGGACCAGCTTGGCAGAACTGTCATGACTTCTCTACCTCCCCCCCTCCCCAC
TGCATGATGTGATGTGGTCAAATTTATATGCATTAATGACCTGGGGGGTCATTAATTAATGTGGAGGGGC
CCCACCCCCCCCCACATCACAGCCCCACCCTGCACATCACAGCCCCGCCCTCCCTCACCAAGCACCTCCC
ACATGCCCATATATGGGCATGATTTGGGCAGCTCTGACCCTGGTCTGTGAGGTCTGGGTCTCTGTGACCT
CACAATGACCAGGGCCCTGCCCGGGTCTATATAAGAGGCCAGGAAGTCGGCCCCTGTCACAGCCCACAAA
TTCCACCTGCTCACAGGTTGGCTGGCTCAACCAAGGCGGTATCCCCTGCTCTGAGCATCCAGGCCGAATC
CACCCAGCACC 3′

A Clontech Genomic Walking kit was used to isolate a promoter sequence from the porcine protamine gene. The two walking primers used based on known sequences were:

(SEQ ID NO: 4)

| | |
|---|---|
| PP1W1: | 5′ GACTTCCTAAAGGATGAGTCAGAGTTGGAGG 3′ |

(SEQ ID NO: 5)

| | |
|---|---|
| PP1W2: | 5′ GGAACAGCAGGTGCTAAGTTCTGAGGCAG 3′ |

A ˜1.0 kb fragment was amplified and sequenced, and the sequence obtained is shown in Table 4. The underlined sequence matches nt 1 to nt 47 of the published sequence. A preferred sequence for use in a porcine targeting construct contains nt 1- nt 694 of the published porcine protamine sequence and 954 bp of sequence obtained in the present invention (bold italics), as shown in Table 5.

TABLE 4

Porcine protamine 1 promoter 5′ flanking sequence
(5′ to 3′) (SEQ ID NO: 6)

5′ GAGAGCTTCTAGAGAAGAGTCTCAAGAACCATACAAA

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

TTTCATGAAAATGGAATCACACACTATGTGCTGCCTCA 3′

TABLE 5

Preferred porcine promoter sequence (5′ to 3′)
(SEQ ID NO: 7)

5′

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

TTTCATGAAAATGGAATCACACACTATGTG
CTGCCTCAGAACTTAGCACCTGCTGTTCCTTCTTCCCAGATGCTGTTCCCTCCTCCAA
CTCTGACTCATCCTTTAGGAAGTCCCTTCACCAGCATTTCCTCAGGAGGCTTTCCTAT
GGCATCCCCTGAGGTCAAGACCCGCCTCCCCAACATACATCCTCATAAAATCTCTGAA
GGTTCTCTCTCTCAGCAATTTTCATGATTATAATTACTCTGTGTGGTCATTTCATTCA
TGTCTCCTGGAGTTAGATTATAAAGTTGACTAGGCAGGAACATGTCTGCCTTGTTTAT
CACTGTATGCAGGGCTTGCCAGAATCTGGCAAACATAGGGGCTCAATAATAATTTGTA
AACTATCCGAGTGAATGAGTGAGTGTCCTTACAGAGGTCACCTCGTGTCCCTCTGCGG
ATGCATCACGGCCCCGCCCTCCCTCACAAGGCCCTCCCACATGCCCATATATGGACAC
GATGCAGGCCGACTCTGGCCCTGGTCTGTGAGGCCTAGGCCTCTGCGACCTCACAATG
ACCAGGGCCCTCCCCGCGTCTATAAGAGGCCCAGCAGTCAGCCCCTGGCACACAGCCT
CCAAAGTTCCACCTGCTCACAGGTTGGCTGGCTCAACCAAGGCGGTATCCCGTTCTAA
3′

The skilled artisan will understand that one or more nucleotides may be deleted, substituted, and/or added to a promoter sequence, while still providing a functional promoter. Preferred promoter sequences are those in which no more than about 2% of the nucleotides differ by deletion, substitution, and/or addition from the sequences disclosed herein; more preferably no more than about 1% of the nucleotides differ by deletion, substitution, and/or addition from the sequences disclosed herein; even more preferably no more than about 0.5% of the nucleotides differ by deletion, substitution, and/or addition from the sequences disclosed herein; and most preferably no more than about 0.1% of the nucleotides differ by deletion, substitution, and/or addition from the sequences disclosed herein. The term “about” in this context refers to ±10% of a given percentage (e.g., about 1% refers to from 0.9% to 1.1%).

Expressed Transgene Sequences

A preferred gene for use in disrupting sperm function is the dominant negative mutant of hamster BiP protein disclosed in Hendershot et al., (1996) Proc Natl Acad Sci USA 93: 5269-5274. This dominant negative mutant has been shown to cause improper protein folding and abnormal expansion of ER in monkey cells (COS cells). Expansion of ER may affect the compaction of sperm head during spermatogenesis and improper folding of sperm surface proteins would disrupt the function and motility of sperm. Since BiP is a native ER protein, it is less likely to diffuse through the cytoplasmic bridges connecting the developing spermatids. If the mutant BiP is expressed in X- or Y-bearing sperm by targeting the BiP cDNA to X or Y chromosome, it may disrupt the function of the sperm population that expresses it. The sequence of wild type hamster BiP is shown below in Table 6. The dominant negative mutant of BiP is identical to the wild type with the exception of a change in the codon at nt 259 from ACC (coding for threonine at amino acid 37) to GGC (coding for glycine). The preferred segment of the gene that was used in the present invention is bounded by the nucleotides indicated in bold underline; the start and stop codons of the coding segment of the gene are indicated in italic underline.

Table 6: Hamster BiP cDNA sequence (5′ to 3′) (SEQ ID NO: 8)

TABLE 6
Hamster BiP cDNA sequence (5′ to 3′) (SEQ ID NO: 8)

GACACTGGCCAAGACAACAGTGACCGGAGGACCTCGCTTTGCGGCTCCGAGAGATCGG
AACGCCGCCGCGCTCCGGGACTACAGCCTGTTGCTGGACTTCGAGACTGCAGACGGAC
CGACCGCTGAGCACTGGCCCACAGCGCCGGCAAGATGAAGTTCCCTATGGTGGCGGCG
GCGCTGCTGCTGCTCTGCGCGGTGCGGGCCGAGGAGGAGGACAAGAAGGAGGATGTGG
GCACGGTGGTCGGCATCGACCTGGGGACCACCTATTCCTGCGTTGGTGTGTTCAAGAA
CGGCCGCGTGGAGATCATAGCCAACGATCAGGGCAACCGCATCACGCCGTCGTATGTG
GCCTTCACTCCTGAAGGCGAGCGTCTGATTGGCGATGCGGCCAAGAACCAGCTCACCT
CCAATCCCGAGAACACGGTCTTCGACGCCAAGCGCCTCATCGGACGCACTTGGAATGA
CCCTTCAGTGCAGCAGGACATCAAGTTCTTGCCTTTCAAGGTGGTTGAAAAGAAAACT
AAACCATACATTCAAGTTGATATTGGAGGTGGGCAAACCAAAACATTTGCCCCAGAAG
AAATTTCTGCCATGGTTCTCACTAAAATGAAAGAAACTGCTGAAGCATATTTGGGAAA
GAAGGTTACCCATGCAGTTGTTACTGTGCCGGCTTACTTCAATGATGCCCAGCGCCAA
GCAACCAAAGATGCTGGCACCATTGCTGGACTGAATGTCATGCGGATCATCAATGAGC
CCACAGCAGCTGCTATTGCGTATGGCCTGGATAAGAGAGAGGGCGAGAAGAACATCCT
CGTTTTTGACCTGGGCGGTGGAACCTTCGATGTGTCTCTTCTGACCATTGACAATGGT
GTCTTTGAAGTGGTGGCCACGAATGGAGACACTCATCTCGGTGGGGAAGACTTTGATC
AGCGGGTTATGGAACACTTCATCAAGCTGTACAAAAAGAAAACTGGGAAAGACGTTAG
AAAAGACAACAGAGCTGTGCAGAAACTTCGTCGTGAGGTGGAAAAGGCTAAGCGAGCC
CTGTCTTCTCAGCATCAAGCAAGAATTGAGATAGAGTCCTTCTTTGAAGGAGAAGACT
TCTCTGAGACCCTGACTCGGGCCAAATTTGAAGAGTTGAACATGGACCTGTTCCGATC
TACCATGAAGCCAGTCCAGAAAGTGTTGGAAGACTCTGATCTGAAGAAATCAGACATT
GATGAAATTGTTCTTGTCGGTGGGTCTACTCGGATTCCCAAGATTCAGCAGCTGGTGA
AAGAGTTCTTCAATGGCAAGGAGCCATCCCGTGGCATAAACCCAGATGAGGCTGTAGC
ATACGGTGCTGCTGTCCAGGCTGGTGTCCTCTCTGGTGATCAAGATACAGGTGATCTG
GTACTGCTTGATGTATGTCCTCTTACACTTGGTATTGAAACAGTGGGAGGTGTCATGA
CCAAACTGATTCCAAGGAACACTGTGGTACCCACCAAGAAGTCTCAGATCTTTTCCAC
AGCTTCTGATAATCAGCCAACTGTAACAATCAAGGTCTATGAAGGTGAACGACCCCTA
ACAAAAGACAACCATCTTCTGGGTACATTTGATCTGACTGGAATTCCTCCTGCTCCTC
GTGGGGTACCCCAGATTGAAGTCACCTTTGAGATAGATGTTAATGGTATTCTTCGAGT
GACAGCTGAAGACAAAGGTACAGGGAACAAAAACAAAATCACAATTACCAATGACCAA
AATCGCCTGACACCTGAAGAAATTGAAAGGATGGTTAATGATGCAGAGAAGTTTGCTG
AGGAAGACAAAAAGCTCAAAGAGCGCATTGATACCAGGAACGAGTTGGAAAGCTATGC
TTACTCTCTCAAGAACCAGATTGGAGATAAAGAAAAGCTGGGCGGTAAACTTTCCTCT
GAAGATAAAGAAACCATGGAGAAAGCTGTAGAGGAAAAGATTGAATGGCTGGAAAGCC
ACCAGGATGCAGACATTGAAGACTTTAAAGCTAAAAAGAAGGAACTAGAGGAAATTGT
TCAGCCTATTATTAGCAAACTCTATGGAAGTGCAGGCCCTCCCCCAACTGGTGAAGAG
GATACATCAGAAAAAGATGAGTTGTAGGTGTACTGATCTGCTAGGGCTGTAATATTGT
AAATATTGGACTCAGGAACTTTCGTTAGGAGAAAATTGAGAGAACTTAAGTCTCGAAT
GTAATTGGAATCTTCACCTCAGAGTGGAGTTGAAAATGCTATAGCCCAAGTGGCTGTT
TACTGCTTTTCATTAGCAGTTGCTCACATGTCTTGGGGTTGGGGAAAGGAGGAATTGG
CAATTTTTAAAATT

Y Chromosome Targeting Sequences

Targeting a specific chromosome site is usually accomplished by insertion of a construct (containing a gene of interest and preferably containing a selectable marker, often neomycin resistance) into a host genome, causing disruption of splicing, promoter function, or reading frame, with or without deletion of the targeted gene. Incorporation of the construct into the genome depends upon insertion into, or replacement of, the endogenous gene by homologous recombination through one or more arms of the construct into one allele of genomic DNA. As starting material, a genomic clone of reasonable length must be obtained from a host genome. For adequate frequencies of homologous recombination, typically at least about 1 kB of uninterrupted sequence is used as a homologous arm, preferably at least about 2 kB, more preferably at least about 4 kB, even more preferably at least about 6 kB, and even more preferably about 7 kB. When more than one homologous arm is used, the arms need not be equal in length (e.g., one arm may contain about 4 kB of sequence, the other about 2 kB). The term “about” as used in this context refers to ±1-% of a given dimension.

To target the transgenic construct to the bovine Y chromosome, a bovine SRY sequence was used as a probe to screen a bovine BAC library to identify sufficient sequence to act as a homologous arm. The primers used for library screening were:

(SEQ ID NO: 9)

| | |
|---|---|
| SRYF3: | 5′ GCA CCT GTG AGA CCC AAG GTT TCA TCT C 3′ |

(SEQ ID NO: 10)

| | |
|---|---|
| SRYR1: | 5′ CAC CTC ATC AGA TTA ATC AGA CAG G 3′ |

In the present invention, a BAC clone containing the bovine SRY gene was isolated, and the genome sequenced towards the 3′ end of the gene. About 11 kB of sequence downstream of SRY on Y chromosome was identified, as shown in Table 7. A 6.6 kB segment of the sequence was used as the homologous arm in the insertion vector, as shown in Table 8.

TABLE 7

An 11 kb sequence 3′ of the bovine SRY gene (5′ to 3′)
(SEQ ID NO: 11)

TTTGAGGCGATTATAACATCCATCCAGTATTTAATTAGCACCTGTGAGACCCAAGGTT
TCATCTCTTTTCTGAAAATTTTCTTTTAATCACTGGCAATAAATACACTTGTTTCCAT
TTTCACTTAAGTTTTGCATTCTTGGAGGGAGAAAACAAAAAATAATAGTGCCTTCATA
TCAAGAATATAAATTATTCAGATTATGTGGCATGGGGGATGGGATAAACAAGATCCTG
TCTGATTAATCTGATGAGGTGTCAGTGAAAATGTAAATCAAAGGTGTTCTAAAAATTT
GCAAATAGGCTAAAGTAGAAAAATTGGCTACGCTTGCAAAGGAAGCATCCCTTTTTTG
GAATGAAAATAAGTCCATGGTGAAACTGTATGATATGATATGATATATATTACATATT
AAAGTCAACTTTCCAATACATATGTTCCAAACTTTTGAAAAACAGTACTTCAAATAAT
AAATCTCAAAACCCAAAACAATATGTAGTGAAATGTGGAGTTTTTGAAAGAAAGTTCA
GGGAAAAAAGGGAGGTAACTTCACAGACTGTTTTATTCCAGGAAAAATATTGTTTAAT
CAGACATTTATGCATTCCAAATGGTAATTATGTGCTATGATAACCTTCTAAACAAATA
CCTCCAGGTTGTATTTTAAAGTATTTCTATATTCTTTCTATTTATATGTATTGGTGTT
TAATATTTTAAGCCTCTGTTTCCACATGTTCATAAATAAACTGTACTTTAACTTTTGT
CAAAATAGGTATGCTTTCCTTTTCTTTAACTTCAAATAAAGGAAAACATATAATACTA
TGTTTAATTTACTTTGCTTTAAATAACATCAGTGACACTGAGTTTGTTTTGGAAATTA
CTATGTACACTTCATCCTGTATACTATAGATGTATAACTGTGTTTCAGGGAGGAAGCT
GGATTCTGATTCCATGTTGGAAATTGTTTCTTTACTTACCTTTTATTTTTATAACCAT
TCTAAATTGCTTGCCTGGGGGACTCTGCCCCTTTTGCTTGACTGTAAACTAAAGTGTC
TTTGTTTTGCTCAAAAAGAAATAGTTTGTCTCTGTTTACCTGTGAATAGAAGAGATTA
ACACACTCCTGAAGACTGGACATTCCCTTGAAGAGGTTTTATAAGACTGAAGATCCTT
CTATTTTATTTTCCCCCTGCCTTTCTCTCTATTCTGGCTTTTGACTTGAGTTCCTCAT
GCTTCTTTTTCTCTGATCTAAACAGAACCTGGTATCCAGACCCTAATAAGATAATTAT
TTTGAGGCACTAGCCTGCCTCTTCTCAGTCTGCCTGCTCTGTGATTAAAGTCTTCTCA
TTGTGTCAACAACTTGTCTCTTGGATTCATTGGCCTGTCATGAGGTGACCAGAGTGAG
CTTGGACTCGGTAACAATTGCACTTTTGGGCTTTAATTATTATGAGTAATGATGTCCT
TTGTACATTTGTATACCCGTCTGTGGTAGAACATTTACAAACATTTCTCCTGAATATA
TGTCTAGGAAAGAAATGAATGGTTTTGTGTATTTTTAGCAAGCTCTTTTTTTTTCTTT
TTTTGTAAATTTTGATGAATATTCTTGTATTTTCTAATGAATATCTGCTATATTTTAA
AATGTGCCAACTTTTAAAAATATTCATTGGTATGAACTAATACCATGAATTCCAGATG
TAATTGGATATGACTCCTTTCTCTACCATTTATCAGGGCTGACATTGATGGATTTGTT
TTGGTCCTCAGTTTGTTTACCTTTAATCAGCAGTAGGAATAATAATAAATTAAAATAA
AAAACAGCAAAGCAGAAAAATAAATCCTCATTGGGATGCTGGGAAGACTATGTAACTT
TAAGGTGTATAATGAATCAATGAGCAAAAATATATAAAGCATTACAATTAAAAGTCAA
CATTAATTAATGCTAACATTAACTAATTAGTATGTTAACTAACACTAACATACTAACA
CTAACTAGTATGGATGATAATTATACAAAATAAAAATGACAAAAGCTACCTACTAGGA
TCTGTGAAAACTAAATAAATGAAGGTCATATTCTGTGAGTGAAAGTATGCATAGTACT
CAGAACAGAGATAAGCACTGGTAACTAACAACTGTTGACTGAAAAATGACAAGAGTTG
TGAATTAAGTTTTCTTTGGGGCAAAATGAGGACTGCAGCCCAGGAGGCAGCATCAGAT
AGCTCTAAGAGACTACTCCAAAGTGGCAGTGGGGGAAAGTCAATATATAAGGTTTTGG
TGAAGGGGGAGTTCAAAACCATGAACTGCTCATTTTACAAGAGGTTTTTTTGTTAGTC
ATGAATATCTGATGTCACCATGAGGGGATTTAGTGCTTACTCTATATATGAGGAGATG
CAAGTATTGAGATCATACAATGTAATCCTAAAGCATCCATCTATCTAAAGACCTGTCT
CGAAGACAGCCTCACCCTGAACTCCCTCAGGGTTGTTGAAGGTCAACAGCATGAGGTT
CAATCACCATAGAGGCAGATGGCAAACACCTTTGTTGTTCAGTTGTCGGCCAATGCTC
TTGATAGATGCCAATTTGTAGTTGACACAACTAATTAACAGAGAAGGCAATGGCAACA
TACTCCAGTACCTCTTGCCTGGAAAATCCCATTGGATGGAAGAGCCTGGTAAGCTGCA
GTTCTATAGGTCGCAAAGAGTCGGACACCACTAGTGACTTACTCTGACTTTTCAGTTT
CAATGCATTGGAGAGAAATGGCACCTACCCCCAATGTTTCTTGGCCTTGGAGAATCCC
AGGGATGGCAGAGCCTGGTGGGCTGCTGTCTGTGGGGTCACAAAGAGTCGGACACAAC
TAAAGCGACTTACCAGCAGCAGCAGCAACAAATTAAAATAATGATAACAAATAACAGT
GACAACCTACCTCAATTGGATGCAGAAAGGACAAAATTACTTAAAGATGCATAATAAT
TCAAGGATTTAAAGTATTTAAAGTACTTAGGGTAGATGTAGGCACTGGTAAAGAAATA
AATAGTTAGAATAATTAAAAAAATAAACAGAAGGACAAAAACAAAAACAAAACCTGTT
CTGCTTCATTGAGATGCTGTGAAGACTGAAGAAACTATGATGCATACAGATTTAATGA
ATATTTAATATATTCAGAGGTTAACATTATTAAAGTGCTAAATAGCAATAATGATAAT
GATGGTAACAATGATAATGATATTATAATAATAAAACCCCTCACTGGAATATTATGAG
ACTAAATAGGTAAAGGTATGTAAGGTTCAAGAAATAAATATACAATGTTCTTACAGTA
AAAGATAACATTAGGAAAGAAGTAATTAATAATAATTACAAATATTAATCATGATAAT
AAATAACAGCAAATCTTTCCTTCAGGGGATTCTGTGAAGACTAAATATGAAAGTATTT
AGATTCAAAGAGTAGATGTATATAATGTACTAAAAATGGAGTTGTTTTATGATGTGTA
GCTATAGCAATAATGAAAGCAACAATGACATCATTTGATATGCCTGTGAAGACTGAAT
AATTTCAAGTGAGCAGAGTTCAAGGAGCACAATGTACTGCAAATTAAGGTCAGTTTTA
ATAGAGAAAAAATCAATACTAATAATAATTCCAATAGCAATAATAGTACAAATATAGC
AATGATGGATACTTAACTAGGATGCTATGAACACTAAGGAAATTAAGACTTAAAGGAT
TTGATGAGAAAGTGTATCTAAAGTACTAAGAGAAGAAAGTCAACATGAGTAAAATCTA
AGTAGTAATAATAATAATTATGAGGATGATGATGATAAAGTAGAAATAAAACCTACTT
CAGGGATGCTGTGAAGACTAAGTGAAGGTGTAGGATTCAAGAAATAAGTATTTTGAAA
TACTTGGAACACCGATAGATATTAGTAAAACACTAATTAATAACACCACCAACATGAA
TAATAATAAATAATAAAAATGAAACACATCATTGGGATACTATGGCAGTTTTTTAACT
AAGTTATGGTATATAGGGGCTGAATGAGTAAATGCATAAAGAAGTACTTAGAAAAGAA
GGATTGGAGACAAGATGGCAGACATTTGTCTGAACGTGAAAGAACACTGAATGAACAC
TGAAAGATGAACTCCCCCAAGTTGGGAGTGACCAATTGCTACTGGAGAAGAGTGGAGA
AGAGCTCAGATGAATGAAGAGGCTGAGTCAAAGCAAAACAACTCCAATGGTGTAAAGA
AAAATATTTCATAGGAATCTGGAATGTTAGGTCCATGAATCAAGATGTTGGGAAGGCT
GGGAGGAGGGAAAGAGAGATTCCATCTTGAAGACTGTCAGTTATCTTAAGGCACGATG
AAAACTGGGCCTGAACCCTGTTAACTATTGTCAAACTAAAGTCAGGAAACTCCATCCT
CACAGATGGCAAAGATTGGAAGTAAAGGTCAGATTGTGTTAGACTAACGATAGTGCCT
GAACGTAAAGGTCAGATTGTGTTAGACTAATGAGAGTGCCTGAACCTGCATGTTGTAG
TTGTTAATTCTTCCACACCTGCATATTGTAAAACAAATTACTAATGTGTAACCAGTTT
GAGTGAACTCTGGGAGTTGGTGATGGACAGGGAGGCCTGGTGTGCTGTGATTAATGGT
GTTGCAAAGAGTCGGACACAACTTAACGACTGAACTGAACTGAACTGAACTGAATGTG
TAACCATTCATGTAGTGGAGGGTATAAAACTGAGTCCTCCAAAATCATCAAGGTCCTT
GTCAGAACCGATTCCCTTGGGCCTGTTATGTGTAATAAAACTGTTCACTATACTGAGT
GTCCTCCAAGGATTGTTCTACAACTCTGGATTCTACAAAATACCTGGTGTGTTGGCTG
TGAAATCCTCAGAGAGAGAGGCACATTGAGCCTCCACCTGAGGCTTTCACTGGGATGA
AAGCTTCTGTGAGGGGATGGCACCTCCTCTCTTAGATCACCTCTTGTTTTATTGACTC
ATCTTTCTAAGCAGACTTCACAAGACTGTGGATTACAGAGGGAAACACTCAAGTAGGT
CCCACTGTAATAGTGGAAGAAGGGGCCTGATCAACTTATTGGGGCTGGATGAACCTGT
GGTGACTGTGTCCTTGTAGGCTCAGTGGGGAATTGTTTACTGAAGTAAGTAAAACACT
GTTAACAGAATCTGTGCCAATTGTTTGTCAATGTCTTACTGGTTTCCAAGCGACTGTC
CAATTTGTGCAACACCCTCCCATTCTCCTAGGCATTCAGGGACTTCCTGAATGTTGTT
TATGACCAGACTGAAACTGAGCTCTGGGTGCACATTTTGTCTGCACTGACCCATAATA
AGACAGACTGGGATCTTGCCAGGATCAGACTTATGCCAAAGAGAAGTATATTGAAAGC
CTTAATAGATTGGATTTGGATGACTGCTAACAGAAAAACATGAGAGATAATACCATGG
AAAGACTTGGTAGGTAAGACCCTTTTCACTTTGATCATAGTTGAGGTACAGTGGCCCT
TGTCCTTCTTTGAGTGGAGACTTCAGTCAGGGCTGGGGTACAAGACCCTAGCAATGAG
CGATGAAATAGAAGTTGGACTTGCTGTAAGTGATAGAAGGAAAGTAAAAAGTAGGAAA
GGTAGCAGAGAATTCAAACCAACCTTTCCTGAATGGATGATTAAAATTTCAAAAGGGT
TTTGGAGGAAACAAACAAGCAAAAAGTGAACCCCAGTCAAGCTTAGAACATTCTGTGA
GCTAGACTGGCCATTCTTTGGGGTAGGATGGCTCTCAGAAGAACCCTATGATTGGACA
AAGTTTGTACTCTGTGGTTACTGAACAGCCAGGACACCCAGACCAGTTCCTGTACTTA
TGGCTACTAACTGCACAAAATCCACATCGTTAGGCCCAGGTATGTTATCTTGGAAAAG
GAGAGAGCAAGATTTTGCTGGTGAAGCAAAATCCAAGAAAGAAAAATTAAAGGAAATA
TGATCTGGAGGAGTAGGAACCTCCCACCATGCCCTCTCATTCTGGAGAACATGAAGGG
GATCCCCAGAAGAAGAGGAAGGGACATTCCTCCTCTGCGTCCCCCAGTGGAGGAGGTT
CTCTCTTCCCTCCTATTGTACCAAAAATCGTGACAGGAGCTACTGCATCAACATTATA
CCCAACCCTCCCCAGTTTAGAAGAGGAGAGAAAGGGAGAGTTAAGTGTTCATAGGCAG
CTGAGGTTCTACAAAGGAACCTCAAGAGAGAGAGGTTTTGCAAATACCTTTTACGGAG
GTTCAAGCAGTATCACAGGTGGGGCCAGATGGGCATATCCATCCTGGCCACACTGTTC
TTTTCTATCAGCCATTCTTTACCACTGGTCTTTTGAACTGGCAAAGGCATACCCCTCC
CTATTCTAAGAAACCATATGGTCAATTCATCGGACAAAGATTATTTTCAGGATCACCA
ACCTGTGTGGGATGACATAGCCCAGCTTCTCCTCACCCTCGTCAGTACAGAAGAAAGA
CACCGGGTCCTCCCAGAAGTATGTAAATGGCTTCACTGGTAAAATTTCAGGTGGGAAT
TTTTTCCTCATTTCTGTGGTGATACCAAAGGGAAGATGAGTGGAAGCTCTAACAGTCA
TCTGGAGGGCTACAGACCCTAGAACCCCTAACAAAGCTGTTTCCTGAAGTATAGGCTA
AAAGCAATCCCCTGGACTTGCTGAAAACCACCCTCTGGTGATAATAGAGCTAAAGGTG
GGAGCCTAGCCCATAAGGAAAAAACAATATCCTATACCACTGGCTGCCAGGGAGGGAA
TCAAGCTTTCACATTGATAGGCTGAAGGGCACAGGCATACTGGTGGAATGCCAATCAC
CATGGAACACCCCTTTCCTCCCAGTAAAGAAGGACAGGGAAAAAGATTATTGGGAAGC
TCAGGGCATGAGATCCCAGCGATGGGTCAGAAAACCCAGCTGACTTGGATTCAGGCTA
CCACAAGGATTCAAAATTTCCTTACAATATTTGGGAGATGCAGACAAAAGAACAAAGC
TAGTGGACGTGATGGAATTCCAGTTGAGCTATTTCAAATCCTGAAAGATGATGCTCTG
AAAGTGAGGCACTCAATATGCCAGCAAATTTGGAAAACTCAGCAGTGGCCACAGGACT
GGAAAAGGTCAGTTTTCATTCCAATCCCAAAGAAAGGCAATGCCAAAGAATGCTCAAA
CTACCGCACAATTACACTCATCTCACACGCTAGTAAAGTAATGCTCAAAATTCTCCAA
GCCAGGCTTCAGCAATACGTGAACTGTGAATTTCCTGATGTTGAAGCTGGTTTTAGAA
AAAGCAGAGGAACCAGAGATCAAATTGCCAACATCTGCTGGATCATGGAAAAAGCAAG
AGAGTTCTAGAAAAATATTTATTTCTGCTTTATTGTCTATGGAAAAGCCATTGACTGT
GTGGATCACAGTACACTGTGGAAAATTCTGAAACAGATGGGAATACCAGACCACTTGA
CCAGCCTCTTGAGAACTCTGTATGCAGGTCAAGAAGTAACAGTTAGAACTGGACATGG
AACAATAGACTGGTTCCAAATAGGAAAAGAAGTACACCAAGGCGGCATATTGTCACCC
TGCTTATTTACCGTGCAGAGTACATGCAGAGTACATCATGAGAAATGCTGGACTGGAA
GAAACACAAGCTGGAATCAAGATTGCAGGGAGAAATATCAATAACCTCTGATATGCAG
ATGACACCACCCTTATGGCAGAAAGTGAAGAGGAACTAAAAAGCCGCTTAAAGAAAGT
GAAAGTGGAGAGTGAAAAAGTTGGCTTAAAGCTCAACATTCAGAAAACGAAGATCATG
GCATCTGGTCCCATCGCTTCATGGGAAAAAGATGGGAAACAGTGTCAGACTTTATTTT
GTTGGGCTCCAAAATCACTGCAGATGGTGAGTGCTGCCATGAAATTAAAAGCACTTAC
TCCCTGGAAGGAAAGTTATGACCAGTTTAGATAGCATATTCAAAACAGAAACATTACT
TTGCCAACAAAGGTCCGTCTAGTCAAGGCTATGGTTTTTCCTGTGGTCATATTTGGAT
GTGAGAGTTGGACTGTGAAAAAGACTGAGCGCTGAAGAATTGATGCTTTTGAACTGTG
GTGTTGGAGAAGACTCTTGAGAGTCCCTTGGACTGCAAAGAGATCCAACTAGTCCATT
CTGAAGGAGATCAGCCCTGGGATTTCTTTGGAAGGAATGATGCTGATGCTGAAACTCC
AGTACTTTGGCCACCTCATGCAAAGAGTTGACTCATTGGAAAAGACTCTGATGCTGGG
AGGGATTGGGGGCAGGAGGAAAATGTGATGACAGAGGATGTGATGTCTGGATGGCATC
ACTGACTCGATAGACATGAGTCTGTGTGAATTCCGGAGTTGGTGCTGGACAGGGCTGC
CTGGTGTGCTGCAATTCATGGGGTTGCAAAGTGTCAGACACAACTGAGCGACTGAACT
GAACTGAACTGAACTGGACCTGGCAACAGATCTCCTCTTCTTCCCATCAGTTACTACT
AAGTGTCAGACCCCACAATGTGTGGATGACCTAGTCCTGATGGCAGAGACTTGTTCTC
AGTGATGGAAAGTGTCAGGAACCACCCAACAATGAGCTGACCTGAGGGAGTGGTGCCC
CAGAAGAATAAAGAAAATGATGACTCTGAAATAAAGTGAGGACCACGGGGCTGATGCC
ATTTACATGCAAAAGCCCAGATCCTAACCCTGCATGCCTTTTATTGTTAACTTCTACT
TCCTTATTTGTGCTCTAGAGATAACTGTTTTTATAATCTCAGATGGAGGGTACAGATA
TACAATGCACAGTCCTGCCTGGTCTACTAAGGGGCAAAGCAAGCAGCCACCCAGGACA
ATAAAGCAAGCAAACTTATGGTGACACCTGTGGTGTCTAGCCTACTAACTACTCAAGT
GACCCCGGTCTTTGACAAGGAAGAAATCACCTGGGCACAAACTGAGTGAGGAACATGG
TGGGAGGATGGATGGTGGAAACTCAGATGGGAGACTTTTTGTCCCCTCTAGGTTGGCA
TTTCAACTCGTTATGAATTTCCATCAATCCATCCATTTAGGTAAAATAAGACTTGCGA
TAATATCTAACTGTCTTGTGTGCAGATGCAAGCTGCCAATGTATAACCTATTCTAAAA
ATAACCTAGTTCCAAGGAGACAGCACCTCCTGGAATTCAATTAAAGAGGACAGCTCTA
TTTAAACATCTACAGGTGGACTTCACTGACATTAAGCCATGCTAAGGATACAAATATT
TGCTGGTGATGGTATGTACATTTCCAGAATGGGTGGAAGTTTATCCCACCAAGACTGA
AAAAACAAGAAAGTGGCCTGATGTATGCTGAGAGACATTATTTGTAGGTTTGAGTTCC
CTTTGAATATAGGATCAGATAATGGGCCTGCATTTATGGTTGAGTTACTTCAACTGGT
TTGCAAAACTGTAAATATTAAATGGAAACTACATACAATGTATAGGCCACAAAGCTCA
GGAATGGTTCAGAAAATGAACTGGGCTATCAAGGTGACTTTGGAAAAATGAGTGTAAG
AAACTGGCACCCCAATCCACCCCCACCCCCATGGATGAACATGCTGTCATTAGCTGCC
ATTAGTGTTAATGAGGATCAGAATCACACTGCCCCCCTCAACAATCTCATGGGTATTC
CCCATATGTGATAATGTTTGGGAGGCCTCCCCCATTTTTCAGAAGTACAGGGAAAATT
ATCATCAAGAGGAAGAATGGAGGTGTTGTGGCAACTGGAATAGTTGGGGAAGCTGATC
CATGATAACCCCTATGTTCAGGAGAGAATTCCATTTTCTCTAGGCACTACTGTACACC
TATACTCATCAGGAGATTTAATGCATAAAGAATTGGAAGCAGCAGACATTGTCCCCCA
TCTGGAAAGGACAACCACAGATCCAGTATGGAGCCACTACTGATGACTCTGCTATTCC
TTTTTTTTTTTTTTTAAATGCTTATCTCTCTCTTTTTTTTTTTTTTAACTTTACATAA
TTGTATTAGTTTTGCCAAATATCAAAATGAATCCGCCACAGGTATACATGTGTTCCCC
ATCCCGAACCCTCTTCCCTCCTCCCTCCCCATACCATCCCTCTGGGCCATCCTAGTGC
ACCAGCCCCAAGCATCCAGCATCATGCATCGAACCTGGACTGGCAACTCGTTTCCTAC
ATGATATTTTACATGTTCATGCCATTCTCCCAAATCTTCCCACACTCTCCAGCTCCCA
CAGAGTCCATAAGACTGTTCTATACATCAGTGTCTCTTTTGCTGTCTCGTACACCAGG
TTATTGTTACCCTCTTTCTAAATTCCATATATATGCGTTAGTATACTGTATTTATGTT
TTTCCTTCTGGCTTACTTCACTCTGTATAATAGGCTCCAGTTTCATCCACCTCATTAG
AACTGATTCAAATGTATTCTTTTTAATGGCTGAGTAATACTCCATTGTGTATATGTAC
CACTGCTTTCTTATCCATTCATCTGCTGATGGACATCTAGGTTGCTTCCATGTCTTGG
CTATTATAAACAGTGCTGCGATGAACATTGGGGTACACGTGTCTCTTTCCCTTCTGGT
TTCCTCAGTGTGTATGCCCAGCAGTGGGGTTGCTGGATCATAAGGCAGTTCTATTTCC
AGTTTTTTAAGGAATCTCCACACTGTTCTCCATAGTGGCTGTACTAGTTTGCATTCCC
ACCAACAGTGTAAGAGGGTTCCCTTTTCTCCACACCCTCTCCAGCATTTATTATTTGT
AGACTTTTGGATCGCAGCCATTCTGACTGGTGTGAAATGGTACCTCATAGTGGTTTTG
ATTTGCATTTCTCTGAAAATGAGTGATGTTGAGCATCTTTTCATGTGCTTGTTAGCCA
TCTGTATGTCTTCTTTGGAGAAATATCTATTTAGTTCTTTGGCCCATTTTTTGATTGG
GTCATTTATTTTTCTGGAGTTGAGCTGTAGGAGTTGCTTGTATATTTTTGAGATTAGT
TGTTTGTCGGTTGCTTCATTTGCTATTATTTTCTCCCATTCTGAAGGCTGTCTGTTCA
CCTTGCTAATAGTTTCCTTTGTTCTTCAGAAGCTTTTAAGGTTAATTAGGTCCCATTT
GTTTATTTTTGCTTTTATTTCCAATGTTCTGTAGGTGGTTCACTGAGGATCCAAGCTT
CACCATGGGAGACGTCACCGGTTCTAGAACCTAGGGAGCTCTGGTACCCACTAGGCGG
CCGCCTAGTGAGTCGTATTACGTAGCTTGGCGTAAT

Preferred homologous arms comprise at least about 1 kB of uninterrupted sequence from Table 7, more preferably at least about 2 kB, even more preferably at least about 4 kB, and even more preferably at least about 6 kB. A particularly preferred 6.6 kb bovine sequence (nt 1461 to nt 8078 of the 11 kb sequence in Table 7) for use as a homologous arm is provided below.

TABLE 8
6.6 kb bovine homologous arm sequence (SEQ ID NO: 12)

GTATACCCGTCTGTGGTAGAACATTTACAAACATTTCTCCTGAATATATGTCTAGGAA
AGAAATGAATGGTTTTGTGTATTTTTAGCAAGCTCTTTTTTTTTCTTTTTTTGTAAAT
TTTGATGAATATTCTTGTATTTTCTAATGAATATCTGCTATATTTTAAAATGTGCCAA
CTTTTAAAAATATTCATTGGTATGAACTAATACCATGAATTCCAGATGTAATTGGATA
TGACTCCTTTCTCTACCATTTATCAGGGCTGACATTGATGGATTTGTTTTGGTCCTCA
GTTTGTTTACCTTTAATCAGCAGTAGGAATAATAATAAATTAAAATAAAAAACAGCAA
AGCAGAAAAATAAATCCTCATTGGGATGCTGGGAAGACTATGTAACTTTAAGGTGTAT
AATGAATCAATGAGCAAAAATATATAAAGCATTACAATTAAAAGTCAACATTAATTAA
TGCTAACATTAACTAATTAGTATGTTAACTAACACTAACATACTAACACTAACTAGTA
TGGATGATAATTATACAAAATAAAAATGACAAAAGCTACCTACTAGGATCTGTGAAAA
CTAAATAAATGAAGGTCATATTCTGTGAGTGAAAGTATGCATAGTACTCAGAACAGAG
ATAAGCACTGGTAACTAACAACTGTTGACTGAAAAATGACAAGAGTTGTGAATTAAGT
TTTCTTTGGGGCAAAATGAGGACTGCAGCCCAGGAGGCAGCATCAGATAGCTCTAAGA
GACTACTCCAAAGTGGCAGTGGGGGAAAGTCAATATATAAGGTTTTGGTGAAGGGGGA
GTTCAAAACCATGAACTGCTCATTTTACAAGAGGTTTTTTTGTTAGTCATGAATATCT
GATGTCACCATGAGGGGATTTAGTGCTTACTCTATATATGAGGAGATGCAAGTATTGA
GATCATACAATGTAATCCTAAAGCATCCATCTATCTAAAGACCTGTCTCGAAGACAGC
CTCACCCTGAACTCCCTCAGGGTTGTTGAAGGTCAACAGCATGAGGTTCAATCACCAT
AGAGGCAGATGGCAAACACCTTTGTTGTTCAGTTGTCGGCCAATGCTCTTGATAGATG
CCAATTTGTAGTTGACACAACTAATTAACAGAGAAGGCAATGGCAACATACTCCAGTA
CCTCTTGCCTGGAAAATCCCATTGGATGGAAGAGCCTGGTAAGCTGCAGTTCTATAGG
TCGCAAAGAGTCGGACACCACTAGTGACTTACTCTGACTTTTCAGTTTCAATGCATTG
GAGAGAAATGGCACCTACCCCCAATGTTTCTTGGCCTTGGAGAATCCCAGGGATGGCA
GAGCCTGGTGGGCTGCTGTCTGTGGGGTCACAAAGAGTCGGACACAACTAAAGCGACT
TACCAGCAGCAGCAGCAACAAATTAAAATAATGATAACAAATAACAGTGACAACCTAC
CTCAATTGGATGCAGAAAGGACAAAATTACTTAAAGATGCATAATAATTCAAGGATTT
AAAGTATTTAAAGTACTTAGGGTAGATGTAGGCACTGGTAAAGAAATAAATAGTTAGA
ATAATTAAAAAAATAAACAGAAGGACAAAAACAAAAACAAAACCTGTTCTGCTTCATT
GAGATGCTGTGAAGACTGAAGAAACTATGATGCATACAGATTTAATGAATATTTAATA
TATTCAGAGGTTAACATTATTAAAGTGCTAAATAGCAATAATGATAATGATGGTAACA
ATGATAATGATATTATAATAATAAAACCCCTCACTGGAATATTATGAGACTAAATAGG
TAAAGGTATGTAAGGTTCAAGAAATAAATATACAATGTTCTTACAGTAAAAGATAACA
TTAGGAAAGAAGTAATTAATAATAATTACAAATATTAATCATGATAATAAATAACAGC
AAATCTTTCCTTCAGGGGATTCTGTGAAGACTAAATATGAAAGTATTTAGATTCAAAG
AGTAGATGTATATAATGTACTAAAAATGGAGTTGTTTTATGATGTGTAGCTATAGCAA
TAATGAAAGCAACAATGACATCATTTGATATGCCTGTGAAGACTGAATAATTTCAAGT
GAGCAGAGTTCAAGGAGCACAATGTACTGCAAATTAAGGTCAGTTTTAATAGAGAAAA
AATCAATACTAATAATAATTCCAATAGCAATAATAGTACAAATATAGCAATGATGGAT
ACTTAACTAGGATGCTATGAACACTAAGGAAATTAAGACTTAAAGGATTTGATGAGAA
AGTGTATCTAAAGTACTAAGAGAAGAAAGTCAACATGAGTAAAATCTAAGTAGTAATA
ATAATAATTATGAGGATGATGATGATAAAGTAGAAATAAAACCTACTTCAGGGATGCT
GTGAAGACTAAGTGAAGGTGTAGGATTCAAGAAATAAGTATTTTGAAATACTTGGAAC
ACCGATAGATATTAGTAAAACACTAATTAATAACACCACCAACATGAATAATAATAAA
TAATAAAAATGAAACACATCATTGGGATACTATGGCAGTTTTTTAACTAAGTTATGGT
ATATAGGGGCTGAATGAGTAAATGCATAAAGAAGTACTTAGAAAAGAAGGATTGGAGA
CAAGATGGCAGACATTTGTCTGAACGTGAAAGAACACTGAATGAACACTGAAAGATGA
ACTCCCCCAAGTTGGGAGTGACCAATTGCTACTGGAGAAGAGTGGAGAAGAGCTCAGA
TGAATGAAGAGGCTGAGTCAAAGCAAAACAACTCCAATGGTGTAAAGAAAAATATTTC
ATAGGAATCTGGAATGTTAGGTCCATGAATCAAGATGTTGGGAAGGCTGGGAGGAGGG
AAAGAGAGATTCCATCTTGAAGACTGTCAGTTATCTTAAGGCACGATGAAAACTGGGC
CTGAACCCTGTTAACTATTGTCAAACTAAAGTCAGGAAACTCCATCCTCACAGATGGC
AAAGATTGGAAGTAAAGGTCAGATTGTGTTAGACTAACGATAGTGCCTGAACGTAAAG
GTCAGATTGTGTTAGACTAATGAGAGTGCCTGAACCTGCATGTTGTAGTTGTTAATTC
TTCCACACCTGCATATTGTAAAACAAATTACTAATGTGTAACCAGTTTGAGTGAACTC
TGGGAGTTGGTGATGGACAGGGAGGCCTGGTGTGCTGTGATTAATGGTGTTGCAAAGA
GTCGGACACAACTTAACGACTGAACTGAACTGAACTGAACTGAATGTGTAACCATTCA
TGTAGTGGAGGGTATAAAACTGAGTCCTCCAAAATCATCAAGGTCCTTGTCAGAACCG
ATTCCCTTGGGCCTGTTATGTGTAATAAAACTGTTCACTATACTGAGTGTCCTCCAAG
GATTGTTCTACAACTCTGGATTCTACAAAATACCTGGTGTGTTGGCTGTGAAATCCTC
AGAGAGAGAGGCACATTGAGCCTCCACCTGAGGCTTTCACTGGGATGAAAGCTTCTGT
GAGGGGATGGCACCTCCTCTCTTAGATCACCTCTTGTTTTATTGACTCATCTTTCTAA
GCAGACTTCACAAGACTGTGGATTACAGAGGGAAACACTCAAGTAGGTCCCACTGTAA
TAGTGGAAGAAGGGGCCTGATCAACTTATTGGGGCTGGATGAACCTGTGGTGACTGTG
TCCTTGTAGGCTCAGTGGGGAATTGTTTACTGAAGTAAGTAAAACACTGTTAACAGAA
TCTGTGCCAATTGTTTGTCAATGTCTTACTGGTTTCCAAGCGACTGTCCAATTTGTGC
AACACCCTCCCATTCTCCTAGGCATTCAGGGACTTCCTGAATGTTGTTTATGACCAGA
CTGAAACTGAGCTCTGGGTGCACATTTTGTCTGCACTGACCCATAATAAGACAGACTG
GGATCTTGCCAGGATCAGACTTATGCCAAAGAGAAGTATATTGAAAGCCTTAATAGAT
TGGATTTGGATGACTGCTAACAGAAAAACATGAGAGATAATACCATGGAAAGACTTGG
TAGGTAAGACCCTTTTCACTTTGATCATAGTTGAGGTACAGTGGCCCTTGTCCTTCTT
TGAGTGGAGACTTCAGTCAGGGCTGGGGTACAAGACCCTAGCAATGAGCGATGAAATA
GAAGTTGGACTTGCTGTAAGTGATAGAAGGAAAGTAAAAAGTAGGAAAGGTAGCAGAG
AATTCAAACCAACCTTTCCTGAATGGATGATTAAAATTTCAAAAGGGTTTTGGAGGAA
ACAAACAAGCAAAAAGTGAACCCCAGTCAAGCTTAGAACATTCTGTGAGCTAGACTGG
CCATTCTTTGGGGTAGGATGGCTCTCAGAAGAACCCTATGATTGGACAAAGTTTGTAC
TCTGTGGTTACTGAACAGCCAGGACACCCAGACCAGTTCCTGTACTTATGGCTACTAA
CTGCACAAAATCCACATCGTTAGGCCCAGGTATGTTATCTTGGAAAAGGAGAGAGCAA
GATTTTGCTGGTGAAGCAAAATCCAAGAAAGAAAAATTAAAGGAAATATGATCTGGAG
GAGTAGGAACCTCCCACCATGCCCTCTCATTCTGGAGAACATGAAGGGGATCCCCAGA
AGAAGAGGAAGGGACATTCCTCCTCTGCGTCCCCCAGTGGAGGAGGTTCTCTCTTCCC
TCCTATTGTACCAAAAATCGTGACAGGAGCTACTGCATCAACATTATACCCAACCCTC
CCCAGTTTAGAAGAGGAGAGAAAGGGAGAGTTAAGTGTTCATAGGCAGCTGAGGTTCT
ACAAAGGAACCTCAAGAGAGAGAGGTTTTGCAAATACCTTTTACGGAGGTTCAAGCAG
TATCACAGGTGGGGCCAGATGGGCATATCCATCCTGGCCACACTGTTCTTTTCTATCA
GCCATTCTTTACCACTGGTCTTTTGAACTGGCAAAGGCATACCCCTCCCTATTCTAAG
AAACCATATGGTCAATTCATCGGACAAAGATTATTTTCAGGATCACCAACCTGTGTGG
GATGACATAGCCCAGCTTCTCCTCACCCTCGTCAGTACAGAAGAAAGACACCGGGTCC
TCCCAGAAGTATGTAAATGGCTTCACTGGTAAAATTTCAGGTGGGAATTTTTTCCTCA
TTTCTGTGGTGATACCAAAGGGAAGATGAGTGGAAGCTCTAACAGTCATCTGGAGGGC
TACAGACCCTAGAACCCCTAACAAAGCTGTTTCCTGAAGTATAGGCTAAAAGCAATCC
CCTGGACTTGCTGAAAACCACCCTCTGGTGATAATAGAGCTAAAGGTGGGAGCCTAGC
CCATAAGGAAAAAACAATATCCTATACCACTGGCTGCCAGGGAGGGAATCAAGCTTTC
ACATTGATAGGCTGAAGGGCACAGGCATACTGGTGGAATGCCAATCACCATGGAACAC
CCCTTTCCTCCCAGTAAAGAAGGACAGGGAAAAAGATTATTGGGAAGCTCAGGGCATG
AGATCCCAGCGATGGGTCAGAAAACCCAGCTGACTTGGATTCAGGCTACCACAAGGAT
TCAAAATTTCCTTACAATATTTGGGAGATGCAGACAAAAGAACAAAGCTAGTGGACGT
GATGGAATTCCAGTTGAGCTATTTCAAATCCTGAAAGATGATGCTCTGAAAGTGAGGC
ACTCAATATGCCAGCAAATTTGGAAAACTCAGCAGTGGCCACAGGACTGGAAAAGGTC
AGTTTTCATTCCAATCCCAAAGAAAGGCAATGCCAAAGAATGCTCAAACTACCGCACA
ATTACACTCATCTCACACGCTAGTAAAGTAATGCTCAAAATTCTCCAAGCCAGGCTTC
AGCAATACGTGAACTGTGAATTTCCTGATGTTGAAGCTGGTTTTAGAAAAAGCAGAGG
AACCAGAGATCAAATTGCCAACATCTGCTGGATCATGGAAAAAGCAAGAGAGTTCTAG
AAAAATATTTATTTCTGCTTTATTGTCTATGGAAAAGCCATTGACTGTGTGGATCACA
GTACACTGTGGAAAATTCTGAAACAGATGGGAATACCAGACCACTTGACCAGCCTCTT
GAGAACTCTGTATGCAGGTCAAGAAGTAACAGTTAGAACTGGACATGGAACAATAGAC
TGGTTCCAAATAGGAAAAGAAGTACACCAAGGCGGCATATTGTCACCCTGCTTATTTA
CCGTGCAGAGTACATGCAGAGTACATCATGAGAAATGCTGGACTGGAAGAAACACAAG
CTGGAATCAAGATTGCAGGGAGAAATATCAATAACCTCTGATATGCAGATGACACCAC
CCTTATGGCAGAAAGTGAAGAGGAACTAAAAAGCCGCTTAAAGAAAGTGAAAGTGGAG
AGTGAAAAAGTTGGCTTAAAGCTCAACATTCAGAAAACGAAGATCATGGCATCTGGTC
CCATCGCTTCATGGGAAAAAGATGGGAAACAGTGTCAGACTTTATTTTGTTGGGCTCC
AAAATCACTGCAGATGGTGAGTGCTGCCATGAAATTAAAAGCACTTACTCCCTGGAAG
GAAAGTTATGACCAGTTTAGATAGCATATTCAAAACAGAAACATTACTTTGCCAACAA
AGGTCCGTCTAGTCAAGGCTATGGTTTTTCCTGTGGTCATATTTGGATGTGAGAGTTG
GACTGTGA

In similar fashion, a porcine SRY gene sequence was used to screen a porcine BAC library to isolate a clone containing the porcine SRY. The primers used for library screening in the present invention were:

| | | |
|---|---|---|
| PSRYF1: | 5′cacctgtgact tagtttcag 3′ | (SEQ ID NO: 13) |
| PSRYR1: | 5′ggctaatcacgggaacaac 3′ | (SEQ ID NO: 14) |

Sequencing downstream from the 3′ end of the SRY gene on the BAC clone, ˜3.8 kb of sequence was obtained. The sequence is shown below in Table 9.

TABLE 9

Sequence 3′ of the porcine SRY gene (5′ to 3′)
(SEQ ID NO: 15)

ACATGTTTGACCTATAAAGAATTACCGGCATGCCAATATGACTCAACCTGTCTTTACG
ACTGCTTAAAAGAGCACTACCTTAATAAGAAAGTATCTTAACACACAAACTGCTTGAT
TTCGAAAACCATCTGTTTTTCCTTCTAATAGAACAATTTTTTTATACCTAATTTTAGT
TGTTCCCGTGATTAGCCATTAAGTACGTAACAGTATATATTAGTATTCTGATAATCCT
TAGCATAGCTGATAGAATTCTCTTTATTCTCACTGTCAAAACTGTAGTGCTGGGGAGC
ATGCACAAATTTATGATACAGGAACTTCCATGGAAGTATTTGTACCTAATAAAGCAGT
CCCTTGTAGAGTCTGTTCTTTTGTCTTTTCAGCTATTTTGCCTGTCTTTGTAAACTGC
AGGTAAAGTAGTGAATATATATGTGTAGTCTATCTGTTTTGAGATTCTTTCTGATATA
TTGCCTCTCCCAGCTTCAGAAGAGAAAGAAGAGTTTTGCTGCCATTTGCAACTCAGTT
CCTTCACTCCGCACAAATCTATGCACTTTGACCTTGAGTTTGAACCATCATGACATCC
TTCTGCTAAGACGAAATCTTTTTCTTCTTCTTTTTAATGAAATCTTTAATTGGCTCCT
GTGAGACTCACGGTTTCGCGTCTTTTCCGAAAGTTTTCTTTTAACCAGGACCAAATGT
TTGTTTCCATTGTCCTCAACTTTGACGTTCTGGGGGGTGCGGGTGGGGAATAGGGATA
TAATGTTGAGAATATGAACTATTCAGATTGGTGGGGAGGGGCGGGGGGAAGGGGGCAT
GGGAGGTGGACGAGCCTGTCCGGTTAATCTGGTGAGAAGTCAGTGAAAATGTAAGTCA
AAGGCATTATAAAATTTGCCTATGGCCTAAAGTAGAAACTCTGGCAGTTTTCAGAGAA
AAGCATCAATTTTTGAAATAAAAATAAGCTGATGGTCTCTTGTCTCTGTATTTATATA
CCATATGCCAAAATTAACTTTCCAGTGCATATATTCCAAAGCTTTAAAAAAAAAAAAA
ATTGTCTCAGGTAGTAAAACTCAAAACAGGAAAATGTATGTGGTAGAGTAAAATGTCA
CGTTTTTGAAAGAAAATACAAGGTAAAACAGGAATTAATTTCACGGACTAATTCGCTC
CAGAAACAGTGCTGTTTATTCGGAGATTTACTGCCCCATCTTCCTCTACCCCCGCCCC
CGCCCCCGCCCCAGGTTGGGAATTATATGTTGCAATAACCTTTTAAACAACTGTCTAA
ACTACTCTTAGGTGGAAACTGTGAACAACAAACCTGCCATAAAAGTATCATCATGAGC
TATGGGTCTGCTCCGGCCTATACTTGTCACCGCTTTGGTACACTTACCGGACATATTT
CTGTCTGTTTAAACTTTGGTCAGCTAAAAATTAAAACTCCCGCCTGGACCAGACCCTA
ACCACCATCCCATGACTACTGACTAGGAGACTCAACACAGGACCCCTGCCCTATAAAA
CTTAACTCTCCCTACACAGCAGGAGGGGTGGGGTGGGGAGGGGCTGAGTTCTTTCTCA
GTGCTCCTGGCCATCTCTTTGGTCAATAAAATTTGTTTGGGACCTCAGTGTTCCAGTT
GACTGTCTTTTCTTCTCTTCTGTGTGTTAACAACTGCTTCGTATTTTTTAATGTCTTT
ATATACATTTTACACACATATATATGCAAACTGACAGTATTAATGGCCTGAACCTAGC
CAGAACTCACATTGGGACTTGAACCCATGATTTTAAATTAGAATCACTCACCCTGTGT
CTGGACTCACTGAGGTTCAGGTTCTTCATGTCTCCTCGCAGAAGGAATTCAGCAAGCG
ACAAAGGGATAGGCAAGAAATAGGTTTATTTTTTTTGGACGCTTGTGAGAGATGCAAG
CAGGCAGGCAAGTTCTGCCCCAAGGATCTGAGGATCCAGATAGATGGGTGGGCTACAG
TTTTATCCTCCAAGGGGAGTGGAGGTGGGAAAAGCCGGCCTTGGTATCCGGTAAGGTG
TGTATTCAAATCAGCAGAAGGGTGGTCCTCAAACTCTTGCCCTTGATCTGAATCTGAA
TGCAGGCCCCATCCCATCTGCACCCAATGACCTGAGGCAATTCTCACACTTCCACTAG
TTAAGCAAGCCTGCCTTGTTCTGATGGCTGTTTTTGAGCAATTTATTTACTTACAGTG
GTCTCCCAATATCCCCTAAGTTTTCCTCTTTATCTGTGGTCCTTTACTGGGACCCCTA
AAACTTCCTGTGCCTACTCCATCCCTATACATATATATATGTATGTATATATGTATAT
GTATGTATTTGTGTTTTATGTTTAAAACCTCTCTTTCTGAAACTGACAGCATTTATGA
CTTGAACCCAGCTAGAGCCCAGACTGGAACTTAACCCACTTTTTTTTCTTTTCTCTTT
TTTTTAAATTACGAATCACTCACCCCGGTGTCCAGCCTTACTTGAGGGTCAGGTTCTT
TGTGCCTCAGCCAACAGAAAGGAATTCAATGGGAGACAAAGTGATAGTCAAGAAACAG
ATTTATTAAGACAGGACGCATGAGAGATGTCAAGTGGGCAGGCAAGGAAGCTCTGCCC
TGAGGCTTAGGTGGGCTACAGTTTTATCCTCAAGGGGAGTGGAGGTCAGAAAAGCCTG
CCTCTTCCTCTTTCTTCCAGTATCTGTTAAGAGAGTGTTTGACCCTGTAAGGTCAAAC
TAGGACTGTCATGGTGCATGTTCACATCAGCAGAAGGGTGGTCCTTAAACTCCTGCCC
TTAGGTCTGAACCTGAATGCAAGCCTCACCACCCCCCCACCCCCTGGCACCCAGTGAC
CTGAGGCAATTCTCATGGCTCCACCACAGGAGAGCAAGCCTGCCTTATTCTGATGGCT
TTCTTGAGCAGTTATTAACTTACAGTGATCTCCCAAAGTTCCCTAGGTTTCCCTCTCT
ATGGTCTTTTAGGACTTTTACAACTACCCGTGTAACTATCCTACTCCATCCCTATCAT
CTCCACATGTACAGAAATAACCTCTACTCAGACCTTCATCAAAAAAGATTGATTTTCC
TTTTATCAAACTTCACATAAATCACAGCATAAAGTATTATGTCAAGTTTGTTGTGCTT
AGTATAATTTCAGTGACATTTCAGCTTGTTTGTCTTAGAAATTACTATGTAATTCCAT
TCTATTTTTTTATAGACATGTGAATGGACACCTTCTGGTTTTAGCACAAGTACATAGT
GTATACATGTCCATGAGAGAACATTTACAGGCATTTCTACTGAGTATATACCTAGGAA
AGAAATTGTGTGTTTTTGACACATATTTAGCAGGTATTGATAACAAATATTTTTGTAT
TTTCTAAAGAATATTTGCCATATCTTCAAATGTCAACATCCAAAAAAATTTAAGGGTA
TGAACTAAGAGGATGGCCTCCAGAGTCAGTCAGTCTGGACCTGACTCCTTCCCTGCCA
TTTATTAGGCCAGACACTGAATGATTGCTTCATCTCTCTGGGCCTCAATTTTCTCACT
TTTAAGTAAGAAGGAGAAGGAGGAGGAGGAAGAGGAGGAGGGGGGAGGAGGAAGGGGG
AGAAGGAGAAAGAGAAGAAGAAGAAGAAGGATAATAATGATAATACTAACGAATGAAA
ATAAAACAATAAGAACAAAACCAACTTCATTGGGATATTGGGAAGACTAAAAAAGTTA
AGGTGTATGATGATTCAATGTACATAAAATATAAAAAGTATATTTAAATAGTATTAAA
GAATTTCCTGTCATGGTGCAGTGGCTAGTGAATTGACGTAGGAACCATGAGGTTGTAG
GTTCAATCCCTGGCCTCGTTCAG

The skilled artisan will understand that one or more nucleotides may be deleted, substituted, and/or added to such a sequence while still providing a functional homologous arm. Preferred homologous arms are those in which no more than about 2% of the nucleotides differ by deletion, substitution, and/or addition from the sequences disclosed herein; more preferably no more than about 1% of the nucleotides differ by deletion, substitution, and/or addition from the sequences disclosed below; even more preferably no more than about 0.5 % of the nucleotides differ by deletion, substitution, and/or addition from the sequences disclosed herein; and most preferably no more than about 0.1% of the nucleotides differ by deletion, substitution, and/or addition from the sequences disclosed herein. The term “about” in this context refers to ±10% of a given percentage (e.g., about 1% refers to from 0.9% to 1.1%).

Transfection and Selection of Transgenic Cells

A day 63 bovine male fetus was collected and the genital ridge cells were obtained by 0.3% protease (Sigma cat. #P6991, St. Loius, Mo.) digestion of the genital ridges for 45 minutes at 37° C. Body cells were obtained from a partial body (minus head and viscera) trypsin-EDTA (Life Technologies cat. #25300-062, Rockville, Md.) digestion for 45 minutes at 37° C. Following digestion and filtration through a 70 μm filter, genital ridge cells were cultured in Amniomax medium (Life Technologies cat. #11269-016) and body cells were cultured in αMEM (Life Technologies cat. #32561-037) supplemented with 10% fetal bovine serum (FBS, Hyclone, Logan, Utah. and 0.1 mM 2-mercaptoethanol.

Prior to transfection by electroporation, cultured genital ridge and body cells were dissociated using trypsin and counted. The insertion vector is linearized by cutting with Avr II, which cuts the vector in the Y chromosome arm piece. An aliquot of genital ridge cells (1.2×10⁷) was pelleted by centrifugation, resuspended in 1.0 ml αMEM without serum and divided into two 0.4 cm electroporation cuvettes (BioRad Laboratories, Hercules, Calif.). To each of these cuvettes was added 50 μg DNA. The cells were subjected to electroporation using 250V and 960 μF (BioRad GenePulser with Capacitance Extender, BioRad Laboratories) and the contents of each cuvette were aliquoted equally into five, 100 mm culture dishes and cultured in Amniomax medium. An aliquot of body cells (1.2×10⁷) was similarly transfected and cultured.

Following 2 days in culture, cells were passaged into selection medium (Amniomax medium containing 600 μg/ml G418, Life Technologies cat. #10131-027). Non-transfected control cells were passaged into selection medium at the same time. Following 14 days of selection, the control cells were dead, while the transfected cells had given rise to drug-resistant colonies. For the genital ridge colonies, the cells were trypsinized, counted and aliquoted into 96-well plates seeding an average of 2 cells per well; drug selection was lowered to 100 μg/ml. The 96-well plates were monitored daily until confluent wells were observed. Typically, cells in these wells were passaged into duplicate wells so that cells could be analyzed and if found to be positive, frozen for future nuclear transfer. In populations of bovine body and genital ridge cells transfected with the mutant BiP-containing vector, PCR analysis indicated that the vector had been incorporated into the genome of the cells.

### Example 2

### Cloning Transgenic Porcine Animals

Porcine Oocyte Recovery and Maturation

Sow and gilt ovaries were collected at separate, local abattoirs and maintained at 30° C. during transport to the laboratory. Follicles ranging from 2-8 mm were aspirated into 50 ml conical centrifuge tubes (BD Biosciences, Franklin Lakes, N.J.) using 18 gauge needles and vacuum set at 100 mm of mercury. Follicular fluid and aspirated oocytes from sows and gilts were pooled separately and rinsed through EmCon® filters (Iowa Veterinary Supply Company, Iowa Falls, Iowa) with HEPES buffered Tyrodes solution (13iowhittaker, Walkersville, Md.). Oocytes surrounded by a compact cumulus mass were selected and placed into North Carolina State University (NCSU) 37 oocyte maturation medium (Petters et al., J Reprod Fertil Suppl 48, 61-73 (1993)) supplemented with 0.1 mg/ml cysteine (Grupen et al., Biol Reprod 53, 173-178 (1995)), 10 ng/ml EGF (epidermal growth factor) (Grupen et al., Reprod Fertil Dev 9, 571-575 (1997)), 10% PFF (porcine follicular fluid) (Naito et al., Gamete Res 21, 289-295 (1988)), 0.5 mg/ml cAMP (Funahashi et al., Biol Reprod 57, 49-53 (1997)), 10 IU/ml each of PMSG (pregnant mare serum gonadotropin) and hCG (human chorionic gonadotropin) for approximately 22 hours (Funahashi et al., J Reprod Fertil 98, 179-185 (1993)) in humidified air at 38.5° C. and 5% CO₂. Subsequently, they were moved to fresh NCSU 37 maturation medium which did not contain cAMP, PMSG or hCG and incubated for an additional 22 hours. After approximately 44 hours in maturation medium, oocytes were stripped of their cumulus cells by vortexing in 0.1% hyaluronidase for 1 minute. Sow and gilt derived oocytes were each used in the in vitro fertilization and nuclear transfer procedures described below. These procedures were controlled so that comparisons could be made between sow and gilt derived oocytes for in vitro embryo development, pregnancy initiation rate upon embryo transfer, and litter size upon farrowing.

Nuclear Transfer

Upon removal of cumulus cells, oocytes were placed in CR2 (Rosenkranz et al., Theriogenology 35, 266 (1991)) embryo culture medium that contained 1 μg/ml Hoechst 33342 and 7.5 μg/ml cytochalasin B for approximately 30 minutes. Micromanipulation of oocytes was performed using glass capillary microtools in 150 μl drops of TL HEPES on 100 mm dishes (BD Biosciences) covered with light mineral oil. Glass capillary microtools were produced using a pipette puller (Sutter Instruments, Novato, Calif.) and microforge (arishige International, East Meadow N.Y.). Metaphase II oocytes were enucleated by removal of the polar body and the associated metaphase plate. Absence of the metaphase plate was visually verified by ultraviolet fluorescence, keeping exposure to a minimum. A single donor cell obtained from a confluent culture by trrpsin-EDTA dissociation was placed in the perivitelline space of the oocyte so as to contact the oocyte membrane. A single electrical pulse of 95 volts for 45 μsec from an ElectroCell Manipulator 200 (Genetronics, San Diego, Calif.) was used to fuse the membranes of the donor cell and oocyte, forming a cybrid. The fusion chamber consisted of wire electrodes 500 um apart and the fusion medium was SOR2 (0.25 M sorbitol, 0.1 mM calcium acetate, 0.5 mM magnesium acetate, 0.1% BSA, pH 7.2, and osmolarity 250). Following the fusion pulse, cybrids were incubated in CR2 embryo culture medium for approximately 4 hours prior to activation.

Activation

Oocytes/cybrids were activated by incubation in 15 μM calcium ionomycin (Calbiochem, San Diego, Calif.) for 20 minutes followed by incubation with 1.9 mM 6-dimethylaminopurine (DMAP) in CR2 for 3-4 hours. After DMAP incubation, cybrids were washed through two 35 mm plates containing TL-HEPES, cultured in CR2 medium containing BSA (3 mg/ml) for 48 hours, then placed in NCSU 23 medium containing 0.4% BSA for 24 hours followed by a final culture in NCSU 23 containing 10% FBS. Total time in culture was for 0-4 days following activation.

Embryo Transfer and Pregnancy Detection

Embryos at various stages of development were surgically transferred into uteri and/or oviducts of asynchronous recipients essentially as described by Rath (Rath et al., Theriogenology 47, 795-800 (1997)). Briefly, recipients (parity 0 or 1 female porcines) were selected that exhibited first standing estrus 24 hours after oocyte activation to 24 hours prior to oocyte activation. For surgical embryo transfer, recipients were anesthetized with a combination of 2 mg/kg ketamine, 0.25 mg/kg tiletamine/zolazepam, 1 mg/kg xylazine and 0.03 mg/kg atropine (Iowa Veterinary Supply). Anesthesia was maintained with 3% halothane (Iowa Veterinary Supply). While in dorsal recumbence, the recipients were aseptically prepared for surgery and a caudal ventral incision was made to expose and examine the reproductive tract. Embryos that were cultured less than 48 hours (1-2 cell stage) were placed in the ampullar region of the oviduct by feeding a 5.5-inch TomCat® catheter (Sherwood Medical) through the ovarian fimbria. Embryos cultured 48 hours or more (≧4 cell stage) were placed in the tip of the uterine horn using a similar catheter. Typically, 100-300 NT embryos were placed in the oviduct or uterine tip, depending on embryonic stage and 100 IVF embryos were placed in the oviduct. All recipients and protocols conformed to University of Wisconsin animal health-care guidelines. Ultrasound detection of pregnancy was accomplished using an Aloka 500 ultrasound scanner (Aloka Co. Ltd, Wallingford, Conn.) with an attached 3.5 MHz trans-abdominal probe. Monitoring for pregnancy initiation began at 23 days post fusion/fertilization and repeated as necessary through day 40. Pregnant recipients were reexamined by ultrasound weekly.

### Example 3

### Cloning Transgenic Bovine Animals

Embryo Construction

Oocytes aspirated from ovaries were matured overnight (about 16-18 hours) in maturation medium. Medium 199 (Biowhittaker, Cat #12-119F) supplemented with luteinizing hormone 10IU/ml (LH; Sigma, Cat #L9773), 1 mg/ml estradiol (Sigma, Cat #E8875) and 10% FCS or estrus cow serum, was used.

Oocytes were stripped of their cumulus cell layers and nuclear material stained with Hoechst 33342 5mg/ml (Sigma, Cat #2261) in TL HEPES solution supplemented with cytochalasin B (7 μg/ml, Sigma, Cat #C6762) for 15 min. Oocytes were then enucleated in TL HEPES solution under mineral oil. A single nuclear donor cell of optimal size (12 to 15 μm) was then inserted from a cell suspension and injected into the perivitelline space of the enucleated oocyte. The cell and oocyte membranes were then induced to fuse by electrofusion in a 500 μm chamber by application of an electrical pulse of 90V for 15 μs, forming a cybrid.

3-4 hours following cybrid formation, cybrid activation was induced by a 4 min exposure to 5 μM calcium ionophore A23187 (Sigma Cat. #C-7522) or ionomycin Ca-salt in HECM (hamster embryo culture medium) containing 1 mg/ml BSA followed by a 1:1000 dilution in HECM containing 30 mg/ml BSA for 5 min. For HECM medium, see, e.g., Seshagiri & Barister, 1989, “Phosphate is required for inhibition of glucose of development of hamster eight-cell embryos in vitro,” Biol. Reprod. 40: 599-606. This step was followed by incubation in CR2 medium containing 1.9 mM 6-dimethylaminopurine (DMAP; Sigma product, Cat #D2629) for 4 hrs followed by a wash in HECM and then culture in CR2 media with BSA (3 mg/ml) under humidified air with 5% CO₂ at 39° C. For CR2 medium, see, e.g., Rosenkrans & First, 1994, “Effect of free amino acids and vitamins on cleavage and developmental rate of bovine zygotes in vitro,” J. Anim. Sci. 72: 434-437. Mitotic divisions of the cybrid formed an embryo. Three days later the embryos were transferred to CR2 media containing 10% FCS for the remainder of their in vitro culture.

Second Nuclear Transfer (Recloning)

Embryos from the first generation NT at the morula stage were disaggregated either by pronase E (1-3 mg/ml in TL HBEPES) or mechanically after treatment with cytochalasin B. Single blastomeres were placed into the perivitelline space of enucleated aged oocytes (28-48 hours of incubation). Aged oocytes were produced by incubating matured “young” oocytes for an additional time in CR2 media with 3 mg/ml BSA in humidified air with 5% CO₂ at 39° C.

A blastomere from a nuclear transfer embryo was fused into the enucleated oocyte via electrofusion in a 500 μm chamber with an electrical pulse of 105V for 15 μs in an isotonic sorbitol solution (0.25 M) at 30° C. Aged oocytes were simultaneously activated with a fusion pulse, not by chemical activation as with young oocytes.

After blastomere-oocyte fusion, the cybrids from the first or second generation NT were cultured in CR2 media supplemented with BSA (3 mg/ml) under humidified air with 5% CO₂ at 39° C. On the third day of culture, developing embryos were evaluated and cultured further until day seven in CR2 media containing 10% FCS. Morphologically good to fair quality embryos were non-surgically transferred into recipient females.

### Example 4

### In Vitro Fertilization

Matured oocytes were inseminated by the procedures described by Long et al. (Theriogenology 51, 1375-1390 (1999)) with a modification described by Grupen and Nottle (Theriogenology 53, 422 (2000)). Briefly, 50 matured oocytes stripped of their cumulus and in a volume of 3 μl, were placed into 92 μl drops of fertilization medium (TLP-PVA). Each drop containing oocytes was inseminated with 5 μl of fertilization medium containing 2000 sperm. Fresh boar semen was purchased from Genes Diffusion (Stoughton, Wis.). Several different boars were used during the course of these experiments. After 10 minutes of co-incubation with sperm, the oocytes were moved to a fresh drop of fertilization medium and incubated for an additional 5 hours. Oocytes were washed through unused fertilization drops to remove sperm and cultured in NCSU 23 with 0.4% BSA until embryos were transferred into recipients 0-4 days post-fertilization. Embryos that were maintained in culture to evaluate development rates were placed in NCSU 23 with 10% FBS from day 5 to day 7.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cell lines, embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. The nucleotide sequences described herein are provided without corresponding homologous sequences according to the Watson/Crick base pairing rules. Those of skill in the art will recognize that the corresponding homologous sequences are also described herein.

Other embodiments are set forth within the following claims.

1. A mammal comprising a transgene on a sex chromosome, wherein the expression of said transgene is operably linked to a promoter region that confers haploid-specific expression to said transgene.

2. The mammal of claim 1, wherein said promoter also confers tissue-specific expression to said transgene.

3. The mammal of claim 2, wherein said tissue-specific expression is testis-specific expression.

4. The mammal of claim 3, wherein said transgene is expressed in one or more cells selected from the group consisting of primary spermatocytes, secondary spermatocytes, spermatids, and spermatozoa.

5. The mammal of claim 1 wherein said promoter region comprises the promoter for the protamine gene.

6. The mammal of claim 1 wherein expression of said transgene selectively kills those cells expressing said transgene.

7. The mammal of claim 1 wherein expression of said transgene selectively disables those cells expressing said transgene.

8. The mammal of claim 1 wherein said transgene encodes a marker protein which can be used to sort those cells expressing said transgene from cells not expressing said transgene.

9. Haploid cells harvested from the mammal of claim 1.

10. Haploid cells according to claim 9 which have been enriched for cells expressing said transgene.

11. The mammal of claim 1 wherein the mammal is an ungulate.

12. The mammal of claim 1 wherein the mammal is selected from the group consisting of porcine, ovine, bovine, and caprine.

13. The mammal of claim 1, wherein expression of said transgene is inducible.

14. The mammal of claim 13, wherein expression of said transgene selectively kills those cells expressing said transgene when exposed to an inducing agent.

15. The mammal of claim 13, wherein expression of said transgene selectively disables those cells expressing said transgene when exposed to an inducing agent.

16. The mammal of claim 13, wherein said transgene encodes a marker protein which can be used to sort those cells expressing said transgene when exposed to an inducing agent from cells not expressing said transgene.

17. Haploid cells harvested from the mammal of claim 13.

18. Haploid cells harvested from the mammal of claim 13 which have been enriched for cells expressing said transgene.

19. A method for producing a population of mammalian haploid cells that is enriched for haploid cells containing a specific sex chromosome, wherein said method comprises:
harvesting haploid cells from a mammal comprising a transgene which is capable of killing or disabling cells in cis when expressed, wherein said transgene is selectively expressed in cells comprising said specific sex chromosome, and wherein the expression of said transgene is operably linked to a promoter region that confers haploid-specific expression to said transgene, whereby expression of the transgene kills or disables those haploid cells expressing said transgene.

20. The method of claim 19, wherein said method further comprises removing or discarding said killed or disabled haploid cells.

21. A method for producing a population of mammalian haploid cells that is enriched for haploid cells containing a specific sex chromosome, wherein said method comprises:
(a) harvesting haploid cells from a mammal comprising a transgene which is capable of killing or disabling cells in cis when expressed, wherein said transgene is selectively expressed in cells comprising said specific sex chromosome, wherein the expression of said transgene is operably linked to a promoter region that confers haploid-specific expression to said transgene, and wherein expression of said transgene is inducible; and
(b) inducing the expression of said transgene to kill or disable the those haploid cells expressing said transgene.

22. The method of claim 19, wherein said method further comprises removing or discarding said killed or disabled haploid cells.

23. A method for producing a population of mammalian haploid cells that is enriched for haploid cells containing a specific sex chromosome, wherein said method comprises
(a) harvesting haploid cells from a mammal comprising a transgene which is capable of generating a detectable phenotype in cells in cis when expressed, wherein said transgene is selectively expressed in cells comprising said specific sex chromosome, and wherein the expression of said transgene is operably linked to a promoter region that confers haploid-specific expression to said transgene, whereby expression of the transgene produces said detectable phenotype marker in those haploid cells expressing said transgene; and
(b) sorting the haploid cells based on the expression of said detectable phenotype.

24. A method for producing a population of mammalian haploid cells that is enriched for haploid cells containing a specific sex chromosome, wherein said method comprises
(a) harvesting haploid cells from an animal comprising a transgene which is capable of generating a detectable phenotype in cells in cis when expressed, wherein said transgene is selectively expressed in cells comprising said specific sex chromosome, wherein the expression of said transgene is operably linked to a promoter region that confers haploid-specific expression to said transgene, and wherein expression of said transgene is inducible, whereby expression of the transgene produces said detectable phenotype in those haploid cells expressing said transgene; and
(b) sorting the haploid cells based on the expression of said detectable phenotype.

25. The method of any one of claims 19-24, wherein the mammal is an ungulate.

26. The method of claim 25 wherein the ungulate is selected from the group consisting of porcine, ovine, bovine, and caprine.

27. The method of any one of claims 19-24, wherein the haploid cells harvested are spermatozoa.

28. A method for producing a mammal, comprising contacting an ovum with one or more spermatozoa produced according to the method of claim 27 to fertilize said ovum.

29. A method according to claim 28, wherein said ovum is fertilized by an assisted reproductive technique.
